(19) United States
(12) Patent Application Publication
(10) Pub. No.: US 2010/0226857 A1
(43) Pub. Date: Sep. 9, 2010

(54) TUMOR CELL DIFFERENTIATION AGENTS AS CHEMICAL INHIBITORS AND TREATMENTS FOR INTRACELLULAR PARASITES

(76) Inventors: Jeannine Strobl, Roanoke, VA (US); David S. Lindsay, Newport, VA (US)

Correspondence Address: LATIMER INTELLECTUAL PROPERTY LAW, LLP, P.O. BOX 711200, HERNDON, VA, 20171, US

(21) Appl. No.: 12/294,794

(22) PCT Filed: Mar. 28, 2007
(86) PCT No.: PCT/US07/07773

§ 371 Date: Sep. 26, 2008

Related U.S. Application Data
(60) Provisional application No. 60/826,113, filed on Sep. 19, 2006.
(60) Provisional application No. 60/786,714, filed on Mar. 29, 2006.

(51) Int. Cl. A61K 49/00; A61K 31/16; A61K 31/473; A61P 33/00; C12Q 1/68

(52) U.S. Cl. 424/9.2; 514/575; 514/296; 435/6

(57) ABSTRACT

The present invention provides new compositions and methods for treatment of intracellular parasites. The compositions comprise one or more hydroxamic acids in an amount sufficient to interfere with the activity of one or more histone deacetylases in the intracellular parasites. The compositions and methods can be used to treat members of the Apicomplexa group of intracellular parasites.

### CROSS-REFERENCE TO RELATED APPLICATIONS

This application relies on and claims the benefit of the filing date of U.S. provisional patent application No. 60/786,714, filed 29 Mar. 2006, and U.S. provisional patent application No. 60/826,113, filed 19 Sep. 2006, the entire disclosures of both of which are hereby incorporated herein by reference.

### BACKGROUND OF THE INVENTION

1. Field of the Invention

The present invention relates to the field of medical and veterinary treatment of diseases and disorders. More specifically, the invention relates to compounds, compositions, and methods for treating subjects infected or otherwise affected by one or more intracellular parasites.

2. Description of Related Art

Infection of animals, including humans, by intracellular parasites is a continuing world-wide problem in medical and veterinary health care. The life cycle of these parasites make them particularly difficult to treat, mainly due to their intracellular nature and the difficulty of eliminating them from their host without causing harmful or irreversible damage to the host. Various compounds and treatments have been proposed and developed for treatment of various intracellular parasites; however, each has a drawback or limitation.

For example, apicidin is a broad-spectrum anti-protozoal agent. It was identified as a histone deacetylase inhibitor by detection of in vitro anti-proliferative activity against the tachyzoite stage of Toxoplasma (Darkin-Rattray et al., 1996). U.S. Pat. No. 6,110,697 to Dulski et al. likewise discloses the use of apicidin and derivatives of it for in vitro inhibition of Toxoplasma proliferation. While these two references show that this compound is active in vitro, in vivo studies are lacking. Subsequently, valproic acid, another histone deacetylase inhibitor, was reported to inhibit increases in T. gondii tachyzoite numbers in vitro (Jones-Brando et al., 2003). Again, in vivo data is lacking on this compound. Thus, although histone deacetylase has been proposed as a drug target for treatment of Toxoplasma infection, the lack of in vivo evidence leaves this an open question. Furthermore, the suitability of histone deacetylase as a target in other intracellular parasites is unaddressed.

Like infection with intracellular parasites, cancers are a significant health problem throughout the world. Among the many compounds that have been found to be useful in treatment of cancers, preliminary clinical successes have been achieved with histone deacetylase inhibitors (see Monneret, 2005). For example, preliminary success was achieved using suberoylanilide hydroxamic acid (SAHA) in cancer treatment trials (Kelly et al., 2005; O'Connor et al., 2006). Other compounds having similar structures, such as scriptaid and trichostatin A (TSA), have shown similar promise. These compounds are believed to act through binding of the compounds to the active site of the histone deacetylase enzyme (Finnin et al., 1999). Even though these compounds showed early promise, of those tested in vivo, many have been found to lack in vivo stability. Thus, derivatives of these compounds are now being tested for effectiveness in cancer therapies.

Although much research has been performed to discover and use compounds for treatment of intracellular parasites, new compounds are needed to provide additional or improved treatments for those infected with such parasites. Indeed, the lack of in vivo studies showing effectiveness of anti-parasite compounds and suitability for use in animals has hampered the advancement of this important field of medical and veterinarian research and development.

### SUMMARY OF THE INVENTION

The present invention provides compounds, compositions, and methods for treating intracellular parasites. In general, the compounds of the invention are suitable for both cancer therapies and for treating intracellular parasites. However, it has been found that many compounds failing in clinical trials for cancer therapies are suitable for use in treating intracellular parasites. That is, the present invention provides the surprising discovery that compounds having anti-cancer activity, which are not particularly well suited for in vivo use for treatment of cancers, are suitable for use in vivo for treatment of intracellular parasites. While not being limited to any particular mode of action, the compounds generally are considered as histone deacetylase (HDAC) inhibitors or reactive oxygen species generators.

Compounds of the invention can comprise one or more families of structurally related compounds that are active as specific inhibitors of growth and/or replication of intracellular parasites, such as, for example, the Apicomplexa (formerly sporozoa) class of intracellular parasites. The compounds can be present alone or in combinations in compositions, which can be used as pharmaceuticals for treatment of animals, including humans. Methods of treatment using the compounds or compositions can treat animals, such as mammals, to reduce, control, or eliminate one or more clinical symptoms of parasite infection, and can result in reduction in numbers or viability of the parasite within one or more cells of the host organism. For example, treatment can cure a subject of an infection.

In a first aspect of the invention, compounds are provided. The compounds have activity against one or more intracellular parasites when in contact with or internalized by the parasites. The activity is seen both in vitro and in vivo. In general, in one family of compounds, the compounds have a core structure represented by Formula I. In another family of compounds, the compounds have a core structure represented by Formula II. As mentioned above, the compounds are effective against intracellular parasites by way of a mechanism that appears to be through inhibition of the activity of an HDAC of the parasite or by release of reactive oxygen species within the parasite cells. While not necessarily so limited, exemplary compounds are: hydroxamic acids, for example, those which are capable of chelating zinc or another metal; and quinolines.

In another aspect of the invention, compositions comprising one or more compounds of the invention are provided. Compositions of the invention can have multiple uses, including, but not limited to, use in research, such as in one or more in vitro assay for growth or infectivity of an intracellular parasite. Likewise, they can be used in vivo to treat a subject (used interchangeably herein with “host”, “person”, “animal”, and “patient”) suffering from an infection with an intracellular parasite. In addition, in another non-limiting example, they can be used in vivo as a prophylactic treatment for subjects that will be exposed to an environment known or suspected of containing one or more intracellular parasites. Where the composition is a pharmaceutical, it can be used to treat, therapeutically or prophylactically, any number of intracellular parasites, including, but not necessarily limited to, members of the Apicomplexa class of parasites, Plasmodium, Babesia, Leucocytozoon, Haemoproteus, Cryptosporidium, Isospora, Cyclospora, Sarcocystis, Besnoitia, Hammondia, and Toxoplasma.

In yet another aspect, methods of treating a subject are provided. In general, the methods comprise administering to a subject in need at least one compound of the invention in an amount sufficient to treat the subject. In embodiments, the amount is an amount sufficient to reduce the severity of at least one clinical symptom of a disease or disorder involving one or more intracellular parasites. In embodiments, the amount is an amount sufficient to reduce the number of intracellular parasites in the host organism a detectable amount. In some embodiments, the methods of treating comprise administering to a subject a composition of the invention, such as a pharmaceutical composition. In general, the amounts of compound(s) administered are in the range of those used in cancer therapies, which is significantly higher than amounts needed to show histone deacetylase inhibition in vitro.

In a further aspect, a method of screening for compounds having activity against one or more intracellular parasites is provided. In general, the method comprises contacting at least one intracellular parasite or sample derived therefrom with a compound of the invention, and determining if the compound has an effect on the viability, growth, or infectivity of the parasite or has an effect on the activity of one or more substances (e.g., enzymes) of the parasite or in a sample derived from the parasite. In embodiments, the compound is comprised in a composition. The method can be practiced in vitro, for example as a screening method for one or more lead compounds or drugs, or can be practiced in vivo, for example to confirm activity of a compound identified through an in vitro assay. Where practiced in vitro, the method may comprise screening multiple compounds at one time, and thus may be a method of high-throughput screening. Of course, one or more positive or negative control reactions may be included to provide the practitioner a better understanding of the effects of the compound(s).

In yet a further aspect, containers and kits are provided, which comprise one or more compounds or compositions of the invention. In general, the containers and kits comprise physically defined spaces for holding, and optionally dispensing, the compound(s) and/or composition(s) of the invention. Containers generally contain sufficient amounts of the compound(s) or composition(s) to perform at least one method of the invention, whether it be an in vitro or in vivo method. Kits can comprise one or more containers, along with some or all of the other substances and equipment for practicing at least one method of the invention. In preferred embodiments, a kit of the invention comprises at least one dose of a pharmaceutical for treating a subject infected with an intracellular parasite.

### BRIEF DESCRIPTION OF THE DRAWINGS

The accompanying drawings, which are incorporated in and constitute a part of this specification, illustrate several embodiments and features of the invention, and together with the written description, serve to explain certain principles of the invention.

FIG. 1 shows the structure of various chemical inhibitors. Panel A: exemplary hydroxamic acids; Panel B: exemplary non-hydroxamic acids; Panel C: general structural features of exemplary compounds of the invention; Panel D: general structural features of additional exemplary compounds of the invention.

FIG. 2 shows a response curve for inhibition of T. gondii tachyzoites by the hydroxamic acid inhibitor Scriptaid (BioMol International, Plymouth Meeting, Pa.). Data are the mean+/−S.D. number of tachyzoites in triplicate wells of a 48-well plate expressed as % of untreated control wells. Untreated wells (100%) are graphed at 10⁻² on the x-axis. The solid curve represents the fit of the data to a sigmoidal curve using Prism GraphPad.

FIG. 3 shows photomicrographs of HS68 cell monolayers. Panel A: 48 hours post-infection with T. gondii tachyzoites (5-6×10⁵ tachyzoites/35 mm² dish); Panel B: uninfected cells; Panel C: infected cells treated 48 hours with 1 micromolar (uM) SAHA; and Panel D: infected cells treated 48 hours with 1 uM scriptaid. Panel E is an enlargement of Panel C to visualize the persistent zoite, a non-proliferative remnant of the T. gondii organism.

FIG. 4 shows a graph comparing the concentration-responses for inhibition of T. gondii tachyzoites for TSA, SAHA, and Scriptaid. Data are the mean+/−SEM of n=4 experiments performed in triplicate.

FIG. 5 shows a graph comparing the in vivo activity of valproic acid, TSA, SAHA, and scriptaid for the treatment of toxoplasmosis in mice.

FIG. 6, Panels A and B, show that there is a dose-dependent response for reactive oxygen species production in cells exposed to NSC3852.

FIG. 7 shows production of reactive oxygen species by TSA, SAHA, and a quinoline compound, 5-nitroso-8-quinolinol (NSC3852) alone or after incubation with MCF-7 cells. In the figure, the zoite is labelled “organelle”.

FIG. 8 shows a graph depicting the anti-Toxoplasma activity of NSC3852, along with the structure of the compound.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description is intended to provide details on certain embodiments and features of the invention, and should not be understood in any way as a limitation on the scope of the invention.

The Apicomplexa class of intracellular parasites includes a large and diverse group (>5000 named species) of organisms. To date, seven species are known to infect humans, while many others are known to infect animals, such as chickens and other agriculturally important animals. Species of Plasmodium are widely recognized as having the greatest impact on human health, including costs incurred to treat and protect against malaria. Species of Cryptosporodium are also important, particularly in water-borne disease and in infections of immunocompromised patients. Many Apicomplexa species are considered opportunistic pathogens, and are thus found in higher numbers in immunocompromised individuals. Several parasites of the Apicomplexa class are important causative agents of animal disease, and are of particular interest in the veterinary medicine and agriculture fields. For example, species of Babesia and Theileria are known to be involved in disease in cattle, while species of Eimeria are known to be involved in disease in poultry.

In recognition of the importance to medical (human) and veterinary (non-human animal) health, the present invention addresses needs in the art for new compounds for treatment of infections of intracellular parasites. The invention recognizes, for the first time, that hydroxamic acid compounds can be used to treat intracellular parasites, using an amount of the inhibitor that is not lethal or highly toxic to the host organism. It also recognizes that quinoline compounds can be used to treat intracellular parasites under the same conditions.

In one aspect of the invention, chemical substances (also referred to herein as “compounds”) are provided. In general, the compounds of the invention have activity against one or more intracellular parasites when in contact with or internalized by the parasites.

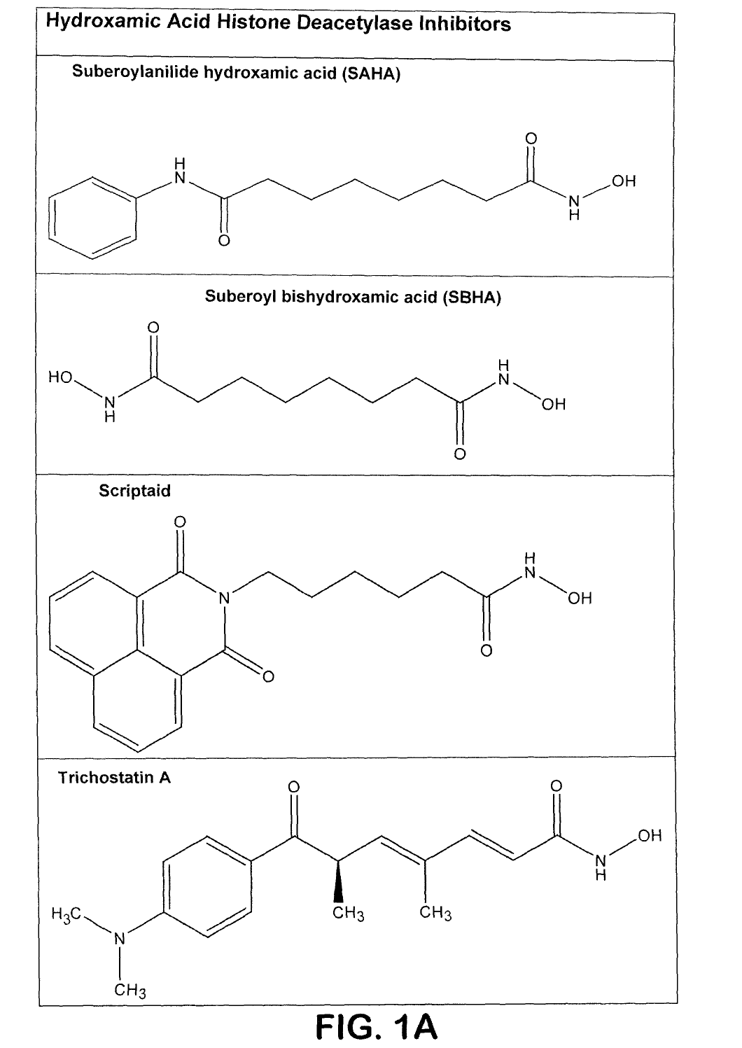

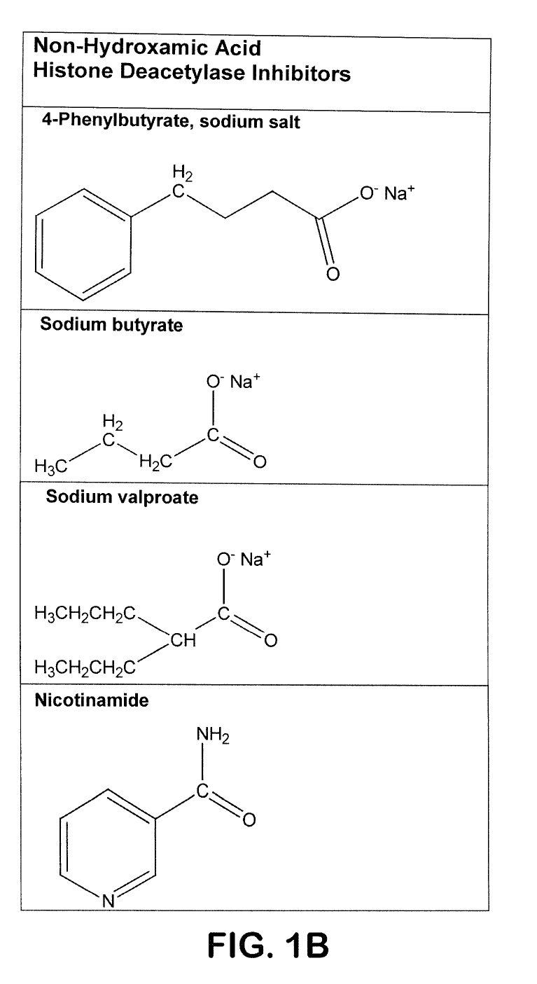

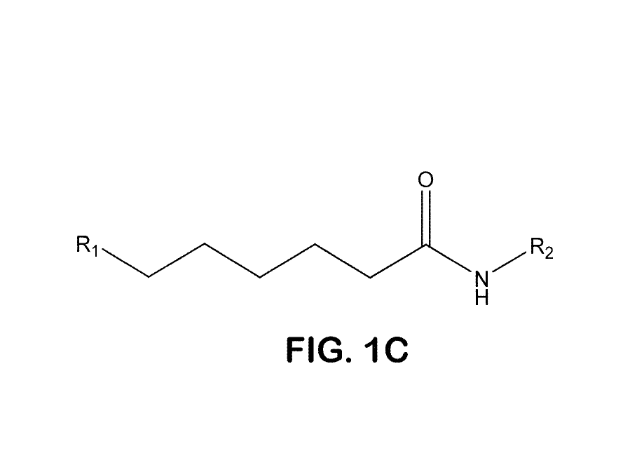

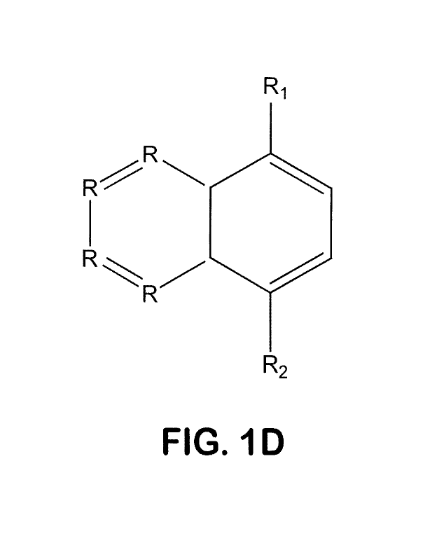

The compounds of the invention fall into two general classes. In one class, the compounds have a core structure represented by Panel C of FIG. 1. In general, these compounds can be represented by hydroxamic acid compounds, or derivatives thereof, such as the compounds of Formula I:

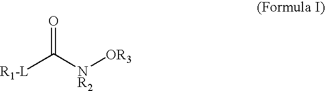

in which:

R₁ may be represented by a nitrogen-containing group or saturated or unsaturated C₃₋₁₄ cycloalkyl or heterocycloalkyl, any of which can be optionally substituted, for example, with alkyl, alkenyl, alkynyl, alkoxy, hydroxyl, hydroxylalkyl, amino, alkylamino, hydroxylamino, alkylcarbonyl, or oxo. For example, R₁ may be represented by substituted or unsubstituted imino or amino, preferably phenylamino or hydroxylamino. Further, for example, R₁ may be represented by phenyl or substituted phenyl, preferably dialkylaminophenyl, such as dimethylaminophenyl. Further, for example, R₁ may be represented by a nitrogen-containing

C₃₋₁₄ heterocycloalkyl, which can be substituted or unsubstituted, preferably saturated C₁₂ heterocycloalkyl, wherein the heteroatom is nitrogen, and wherein the heterocycle is substituted with oxo.

L may be represented by a C₃₋₈ hydrocarbon chain, which can be unsaturated, saturated, straight, or branched, and optionally substituted, for example, with C₁₋₄ alkyl, C₂₋₄ alkenyl or alkynyl, or oxo. For example, L may be represented by straight or branched C₃₋₄ alkyl, preferably C₇ alkyl substituted with oxo. Further, for example, L may be represented by straight or branched C₃₋₈ alkenyl or alkynyl optionally substituted with alkyl or oxo, preferably C₆ alkenyl substituted with methyl and oxo.

R₂ may be represented by hydrogen, alkyl, alkenyl, alkynyl, alkoxy, hydroxylalkyl, hydroxyl, or an amino protecting group. Preferably, R₂ is represented by hydrogen.

R₃ may be represented by hydrogen, alkyl, hydroxylalkyl, or a hydroxyl protecting group. Preferably, R₃ is represented by hydrogen.

In some embodiments, the compounds of the invention are represented by the formulas provided in the figures, and may be referred to as SAHA and scriptaid, for example. Other non-limiting examples of compounds are TSA, trichostatic acid, depudecin, CHAP31, trapoxin A, trapoxin B, K-TRAP, oxamfiatin, tubacin, HC-toxin, and histacin.

In the other class of compounds according to the invention, the compounds have a core structure represented by Panel D of FIG. 1. In general, these compounds can be represented by hydroxamic acid compounds, or derivatives thereof, such as the compounds of Formula II:

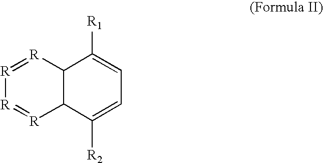

in which

R is N or C, wherein only one R per molecule may be N and the remaining R are C;

R₁ is a hydrogen, alkyl, alkenyl, alkynyl, alkoxy, hydroxylalkyl, hydroxyl, amino, nitroso, or sulfur-containing group, where preferably R₁ is a hydroxyl (OH) or nitroso (N═O) group;

R₂ is a hydrogen, alkyl, alkenyl, alkynyl, alkoxy, hydroxylalkyl, hydroxyl, amino, nitroso, or sulfur-containing group, where preferably R₂ is a hydroxyl (OH) or nitroso (N═O) group.

Preferably, where R₁ is a hydroxyl group, R₂ is a nitroso group, and vice versa.

While not being limited to any specific mode of action, compounds of the invention can chelate zinc and other divalent metal ions, produce reactive oxygen species, and act as histone deacetylase inhibitors. All of these actions, and each one independently, are expected to exert their effects within the cells of intracellular parasites. While not necessarily so limited as a class, exemplary compounds are hydroxamic acids, for example, those which are capable of chelating zinc or another metal. Other non-limiting exemplary compounds are those of the quinoline class, which also chelate zinc and can act as histone deacetylase inhibitors and generators of reactive oxygen species. Those of skill in the art are capable of identifying numerous compounds falling within the broad structures provided herein. Accordingly, each structure need not be individually provided and each compound need not be individually named for those of skill in the art to comprehend them. Assays for determining activity are also well known in the art, and thus may be practiced by those of skill in the art without undue experimentation to identify compounds according to the invention that are not specifically recited.

It is to be understood that the compounds of the invention function, at least in part, by way of interference with maintenance and copying of intracellular parasite DNA. Typically, the compounds cause DNA damage in the genomic DNA of intracellular parasites, reducing or destroying the viability of the organisms by interfering with replication of the genome. Often, the intracellular parasites are killed by induction of an apoptotic pathway as a result of the presence and activity of the compounds of the invention.

It is thus important to understand that the compounds of the invention might have other, possibly known functions. However, they are not known in the art as having DNA damaging activity. Indeed, some of the compounds of the invention, which are exemplified herein, are understood in the art to function as inhibitors of the histone deacetylase function of histone deacetylase enzymes (HDAC). While it is possible that data supports such a function, typically compounds of the invention that are known for that function are instead functioning within the present invention by way of a separate and distinct mechanism. Accordingly, compounds that are known as having HDAC activity do not necessarily function according to the present invention as control agents for intracellular parasites.

For example, a compound of the invention that is known to inhibit the histone deacetylase function of an HDAC can, according to the present invention, inhibit another function of that enzyme (e.g., inhibit cytoskeletal formation and maintenance), or can function in a manner that is completely independent of HDAC inhibition. Among other things, compounds of the invention may function to kill intracellular parasites by causing generation of reactive oxygen species within the parasites, resulting in DNA damage and cell death.

In another aspect of the invention, compositions comprising one or more compound of the invention are provided. In general, a composition of the invention comprises at least one compound according to the invention. Where there is a single compound present, the composition comprises another substance, such as a compatible solid or liquid (e.g., water, salt, binder, excipient, etc.). Typically, the compositions comprise a sufficient amount of the compound(s) to achieve a particular goal. For example, where the composition is for use in an in vitro assay, a sufficient amount of one or more compounds of the invention are provided in the composition to perform at least one assay. Alternatively, for example, where the composition is for use in vivo, for example for treating a subject infected with an intracellular parasite, the composition comprises a sufficient amount of one or more compounds to provide at least one dose to the subject. In embodiments, the amount exceeds the amount needed for one use.

Compositions of the invention can have multiple uses, including, but not limited to, use in research, such as in one or more in vitro assay for growth or infectivity of an intracellular parasite. Likewise, they can be used in vivo to treat a subject suffering or suspected of suffering from an infection with an intracellular parasite. In addition, in another non-limiting example, they can be used in vivo as a prophylactic treatment for subjects that will be exposed to an environment known or suspected of containing one or more intracellular parasite.

In view of the usefulness of the compounds and compositions of the invention in treating subjects, the invention provides pharmaceuticals (also referred to herein as drugs). In general, pharmaceutical compositions according to the invention comprise an amount of one or more compounds of the invention sufficient to treat at least one patient infected with one or more intracellular parasites. Preferably, treatment provides an improvement in at least one clinical symptom of a disease or disorder, and/or a reduction in the number of viable parasites in the host organism. In embodiments, the pharmaceutical composition comprises sufficient compound(s) for two or more doses.

The pharmaceuticals also typically comprise one or more other substances, which are pharmaceutically acceptable. The term “pharmaceutically acceptable” is used herein to mean any substance that is, within the scope of sound medical judgment, suitable for use in contact with the tissues of human beings and animals without excessive toxicity, irritation, allergic response, or other problem or complication, commensurate with a reasonable benefit/risk ratio. For example, such substances can be water or other liquid carrier, such as saline, an organic solvent or mixture of water and organic solvent; solid carriers, such as: fillers; colorants; sugars, such as lactose, glucose, and sucrose; starches, such as corn starch, potato starch, and substituted or unsubstituted beta-cyclodextrin; cellulose, and its derivatives, such as sodium carboxymethyl cellulose, ethyl cellulose, and cellulose acetate; gelatin; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; buffering agents, such as magnesium hydroxide and aluminum hydroxide; biologically active agents; and the like.

Pharmaceuticals may also comprise one or more pharmaceutically acceptable salts. By this, it is meant non-toxic acid addition salts of one or more of the biologically active ingredients. Non-limiting examples of such salts are those comprising: hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, amino acid salts, and the like. Those of skill in the art are cognizant of means for making such salts, and thus the techniques need not be described herein. Included among the methods are reacting acidic compounds of the invention with bases to form salts, or derivatizing compounds of the invention to comprise acid moieties, which may be reactive to form salts when exposed to a base.

Other pharmaceutically acceptable substances include wetting agents, emulsifiers, lubricants, release agents, coatings, perfumes, and preservatives such as antioxidants.

The pharmaceuticals may be provided in any suitable form, including, but not limited to pills, capsules, caplets, solutions, gels, lozenges, injectables, infusibles, suppositories, topical emulsions, and the like. For example, compositions suitable for oral administration may be in the form of capsules, cachets, pills, tablets, lozenges, powders, granules, sprays, or as a solution or a suspension in an aqueous or non-aqueous liquid, such as an elixir or syrup. Compositions for delivery by way of mucous membranes, such as by rectal or vaginal administration, may be formulated as a liquid or suppository in the form of tampons, creams, gels, pastes, foams, powders, or spray formulations. Compositions for delivery by way of topical or transdermal administration include powders, sprays, ointments, pastes, creams, lotions, gels, solutions, patches, and sprays. Parenteral delivery can be by way of aqueous solutions comprising a compound of the invention. Those of skill in the art are well aware of techniques for forming and delivering such pharmaceutical composition, and thus such techniques need not be disclosed herein.

Within pharmaceutical compositions, the compounds of the invention may be the only active ingredients, or may be one of multiple active ingredients. Further, while not so limited, often, pharmaceutical compositions will be prepared in a single dosage form. In general, the amount of active ingredient that is combined with a carrier material to produce a single dosage form will be an amount that is sufficient to produces a therapeutic or prophylactic effect. Of course, multiple doses may be required to achieve a full clinical outcome. In such situations, multiple compositions according to the invention can be provided, each of which may be the same as all others in form and content, or differences may exist between various compositions. For instance, in some situations, more of the active ingredient might be desired in the first dose than in subsequent doses, or vice versa.

The pharmaceutical of the invention can be used to treat, therapeutically or prophylactically, any number of intracellular parasites, including, but not necessarily limited to, members of the Apicomplexa class of parasites, such as Plasmodium, Babesia, Cryptosporidium, Isospora, Cyclospora, Sarcocystis, and Toioplasma. For example, it can be used to treat P. falciparum, P. vivax, P. malariae, and P. ovale. Likewise, it can be used to treat T. gondii, T. cruzi, and T. brucei. In addition, it can be used to treat C. parvum and C. hominus. Leishmania species, such as L. amazonesis, L. donovani, L. infantum, and L. mexicana can also be treated, as can Entamoeba species, such as Entamoeba histolytica and Entamoeba invadens. In a similar fashion, Giardia species, such as Giardia lamblia can be treated. Other intracellular parasites, such as those of the Apicomplexa class of parasites, can be treated as well, such as E. tenella, S. neurona, N. caninum, and/or N. hughesi, as can the fungus Pneumocystis carinii.

Accordingly, the invention provides for the use of one or more compounds or one or more compositions of the invention for the production or manufacture of a pharmaceutical or medicament to treat a person infected with an intracellular parasite (use in manufacturing a therapeutic) or who will soon be in an environment containing one or more intracellular parasites (use in manufacturing a prophylactic). It likewise provides for the use of the pharmaceutical in treating, therapeutically or prophylactically, a subject in need thereof.

In yet another aspect, methods of treating a subject are provided. In general, the methods comprise administering to a subject in need at least one compound of the invention in an amount sufficient to treat the subject. For example, the method may comprise administering a composition comprising one or more compounds of the invention. By administration, it is meant any act that results in a compound or composition of the invention contacting the surface or interior of at least one cell of a multicellular animal. For example, it can comprise exposing an animal, such as a human or other mammal, a chicken, turkey, or other bird, or a cow, steer, or other bovine, to a compound or composition of the invention such that the compound or composition contacts a cell of the animal. Administering thus may include, but is not limited to, injecting, infusing, dissolving, diffusing, swallowing, inhaling, dropping or dripping, spraying, and rubbing. Accordingly, the compound may be formulated in any suitable form, such as an injectable, an infusible, a topical (e.g., cream, salve, lotion, ointment), a dissolvable (e.g., suppository, lozenge), an inhalant (e.g., powder, liquid, aerosol), and the like (see above).

As a general matter, the amount to be administered is an amount sufficient to achieve a desired effect. Thus, it may be an amount sufficient to reduce the severity of at least one clinical symptom of a disease or disorder involving one or more intracellular parasites. It likewise may be an amount sufficient to reduce the number of intracellular parasites in the host organism a detectable amount (e.g., 1%, 5%, 10%, 50%, 75%, 90%, 95%, 99%, greater than 99%, or completely). The desired affect may be achieved over any period of time, but is typically judged in consideration of progression of a particular disease, such as in terms of the typical time for a disease to increase or decrease a detectable amount based on clinical symptoms. Those of skill in the art may determine particular amounts to administer to a particular animal based on the information provided in the Examples, below, and common principles known in the medical and veterinary arts.

Actual dosage levels of the active ingredients in the pharmaceutical compositions of this invention may be varied so as to obtain an amount of the active ingredient which is effective to achieve the desired therapeutic response for a particular patient, composition, and mode of administration. The amount of active agent in a pharmaceutical composition, and the dosing regimen, will thus vary depending on numerous factors. Among the many factors to consider are age, sex, weight, and general health of the patient; the pharmacokinetics (e.g., solubility, specific activity, bioavailability, clearance rate) of the particular compound used; the route of administration; targeting of the pharmaceutical (i.e., local or systemic); and the nature, severity, and stage of progression of the infection.

Although it will be well recognized by those of skill in the medical and veterinary arts that precise doses and dosing regimens will vary, in general, a compound according to the present invention may be administered in an amount to achieve DNA damage in the cells of intracellular parasites, but little or no damage to the host cell DNA. The in vivo model of murine toxoplasmosis has allowed us to define effective doses for suppression of highly aggressive RH strain of T. gondii as about 50 mg/kg/day (in a 20 g mouse this is a 1 mg/mouse dose) given as daily intraperitoneal doses in a DMSO solution for 14 sequential days. For better control, a dose up to 100 mg/kg/day can be tolerated for this duration. The daily dose of scriptaid that can extend the life of mice infected with RH T. gondii is about 3.5 mg/kg for 14 days. This is a non-toxic dose to the animal. Treatment ranges between 3.5 mg/kg and 25 mg/kg scriptaid are considered therapeutic in mice.

With regard to SAHA, useful doses can be up to 6 mg/kg human or animal body mass per day, but will typically be less, such as 10-fold or 100-fold or more less. For example, in the case of oral administration of SAHA for the treatment of intracellular parasites in humans, various schedules can be provided. In a first non-limiting example, the regimen can comprise three or four weeks of treatment comprising administering SAHA at 1-400 mg orally once a day on 3-5 consecutive days/week. In another non-limiting example, the regimen can comprise three or four weeks of treatment comprising administering SAHA at 1-200 mg orally twice a day (b.i.d.) every day of the week. In yet a further non-limiting example, a regimen can comprise three or four weeks of treatment comprising orally dosing twice a day (b.i.d.) with 1-300 mg on 3-5 consecutive days/week.

For veterinary applications of SAHA in the treatment of intracellular parasites, the human doses and schedules described above will be therapeutic, particularly at the higher range of doses listed. SAHA has broad applicability to treatment of Toxoplasmosis in felines, including house cats. Oral dosing of feline pets with SAHA can reduce risk of Toxoplasmosis transmission to humans. Further, we note the usefulness of SAHA as a feed supplement to pigs or other animals raised for human consumption, at the oral doses described, to reduce or eliminate Toxoplasmosis infections in animals, and in particular pork. Handling or consumption of Toxoplasmosis-contaminated pork is a major route of transmission of Toxoplasmosis to humans. Thus, the therapeutic potential of SAHA (and other hydroxamic acid and quinoline inhibitors) extends to prevention of human Toxoplasmosis by reducing zoonotic transmission of the disease as well as direct therapeutic application of the inhibitors to humans afflicted with Toxoplasmosis.

Furthermore, therapeutic doses of TSA for human and veterinary use are provided by the invention. It can be determined that the dosages of the hydroxamic acid inhibitor TSA needed for inhibition of human cancer cells in vitro are toxic in humans. However, intracellular parasites, e.g., RH strain T. gondii, are relatively more sensitive to TSA than human normal fibroblasts or tumor cells. Therefore, administration, such as parenteral administration, of TSA to humans for the treatment of intracellular parasitic disease can be accomplished. For example, parenteral administration of TSA can by: daily doses of 0.5-150 mg for three to four weeks, or daily doses of 0.5-200 mg/day for three consecutive days/week for three to four weeks. The TSA doses recommended for veterinary use is daily intraperitoneal injection of about 0.01 mg TSA/20 g body weight (0.5 mg/kg) for 15 days. Alternatively, the use of 2 mg/kg, 5 mg/kg, or 10 mg/kg body weight given for either 1, 2, or 3 sequential days can be effective, while being tolerated in animals.

Therapeutic doses of scriptaid for human and veterinary use are also provided herein. Scriptaid is the most potent of the hydroxamic acid inhibitors we tested on RH strain T. gondii. Mice readily tolerated a daily scriptaid dose of 3.5 mg/kg by intraperitoneal injection. Based on this data, among other things, a therapeutic range of scriptaid for use in the treatment of human intracellular parasitic disease can be: once daily parenteral administration of 500-1500 mg (7-21 mg/kg based upon a 70 kg patient) for three to four weeks; or once or twice daily oral administration of 500-2500 mg for three to four weeks. For veterinary use, a therapeutic dose of 7-50 mg/kg given as daily intraperitoneal injections for 14 days or oral doses of 7-2500 mg once or twice daily for three to four weeks is recommended.

In contrast to the compounds of the present invention, the histone deacetylase inhibitor valproic acid at very high doses up to 600 mg/kg/day (or 12 mg/20 g mouse/day) was not therapeutic against RH T. gondii. Valproic acid treatment by oral or intraperitoneal routes (twice daily treatments) were ineffective in restricting RH T. gondii infections in mice. Thus, there is a significant difference in activity among HDAC inhibitors, with those that create DNA damage being by far more active, and suitable of use in vivo for treatment of intracellular parasites.

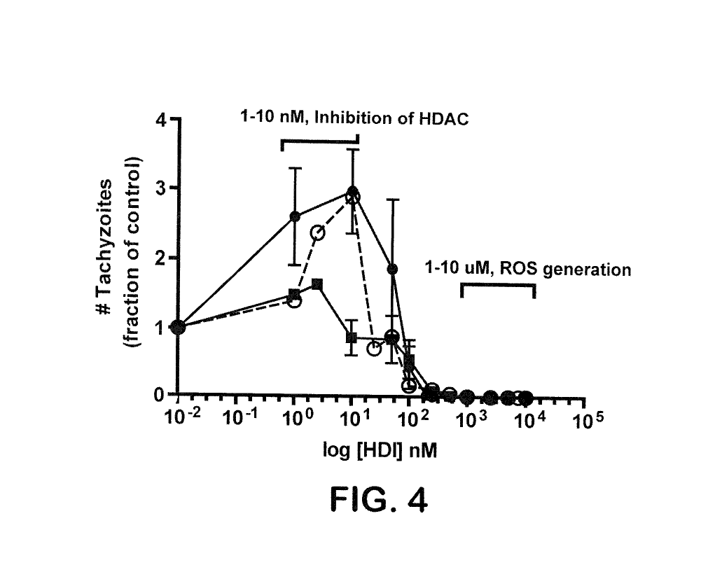

As a general matter, therapeutic or prophylactic doses and dosing regimens according to the present invention will be in the range of those for treatment of cancers in vivo with compounds that cause DNA damage. As can be seen in FIG. 4 (discussed in more detail below), the amounts of compounds according to the present invention that are useful for inhibition of growth of intracellular parasites is considerably higher than the amounts of inhibitors of HDAC that have been reported in the literature. Indeed, the amounts disclosed in the literature as being useful for inhibiting HDAC in vitro stimulate intracellular parasite growth in vivo. In some embodiments, the invention excludes treatments using certain HDAC inhibitors, such as apicidin and TSA, at concentrations reported in the art as being useful for inhibiting the histone deacetylase activity of an HDAC in vitro.

In a further aspect, a method of screening for compounds having activity against one or more intracellular parasites is provided. In general, the method comprises contacting at least one intracellular parasite or sample derived therefrom with a compound, and determining if the compound has an effect on the viability, growth, or infectivity of the parasite or has an effect on the activity of one or more substances (e.g., enzymes) of the parasite or in a sample derived from the parasite. In embodiments, the compound is a compound of the invention. In embodiments, the compound is comprised in a composition. As used herein, the act of contacting can be any act that results in contact of at least one molecule of the compound with at least one intracellular parasite. Thus, contacting may comprise exposing a multi-cellular animal to a compound, allowing the compound to enter the animal's body and cells, and contact an intracellular parasite within a cell of the animal.

The method of screening can be practiced in vitro, for example as a screening method for one or more lead compounds or drugs. Where practice in vitro, the method can comprise adding a compound or composition, such as one comprising a compound of the invention, to an in vitro environment comprising at least one intracellular parasite or an environment comprising biological material derived from at least one intracellular parasite. For example, it may comprise a cell lysate of an intracellular parasite, or a fraction of such a cell lysate. The in vitro environment thus may be an enzyme assay mixture, with some or all of the reagents and substances needed for performance of the assay. Where practiced in vitro, the method may comprise screening one or multiple compounds at one time, and thus may be a method of high-throughput screening. As with practice in vivo, one or more positive or negative control reactions may be included to provide the practitioner a better understanding of the effects of the compound(s).

Alternatively, the screening method can be practiced in vivo, for example to confirm activity of a compound identified through an in vitro assay or as an initial screening in a model organism. Practice in vivo can comprise exposing at least one cell that is infected with an intracellular parasite to at least one compound, and determining the effect of the compound(s) on the growth of the cell or the parasite. Numerous assays for growth of host cells and/or intracellular parasites are known in the art, and any suitable assay may be used.

For example, a screening method may comprise infecting an animal with an intracellular parasite, allowing the parasite to grow within the cells of the animal, introducing a compound of the invention into the animal, and determining if the compound had an detectable effect on the animal or the parasite. In embodiments, the step of introducing can be repeated. According to the method, introducing can be any act that results in the compound contacting a cell of the animal. It thus may be administering, as discussed above. A detectable effect on the animal can be any measurable effect, but will typically relate to the health or viability of the animal, or the number of intracellular parasites that can be detected in the animal. A detectable effect on the parasite can likewise be any measurable effect. Typically, the effect will be viability or total number of cells in an animal in which the parasite is living. Detection can be by way of any suitable biochemical or biological assay, and can either be performed after sacrificing the animal or without the need to sacrifice the animal or seriously harm the animal. For example, it can be performed on tissue biopsied from the animal.

### EXAMPLES

The invention will now be further explained by the following Examples, which are intended to be purely exemplary of the invention, and should not be considered as limiting the invention in any way. It is to be understood that the concepts and results described in the following Examples can be extended beyond the particular compounds and organisms specifically described to other, similar compounds and related organisms.

Toxoplasma gondii is a well-recognized cause of disease in congenitally infected and immunocompromised individuals. The present invention provides detailed studies that show the activity of hydroxamic acid compounds (also referred to herein as hydroxamic acid inhibitors) against the RH strain of T. gondii growing in HS68 human foreskin fibroblast cells. The results show that nanomolar (nM) concentrations of suberoylanilide hydroxamic acid (SAHA), suberic bishydroxamic acid (SBHA), scriptaid, and trichostatin A (TSA) inhibited T. gondii tachyzoite proliferation. The results show that scriptaid was the most potent hydroxamic acid inhibitor (IC₅₀=37 nM). In comparison, the carboxylate inhibitors sodium valproate, sodium butyrate, and 4-phenylbutyrate were less potent (IC₅₀ range 1-5 millimolar (mM). All of the inhibitors tested except SBHA completely protected the HS68 monolayers from T. gondii at concentrations 3-6 times greater than their respective IC₅₀. In contrast, nicotinamide had minimal activity against T. gondii in in vitro assays. We conclude that hydroxamic acid inhibitors exhibit potent anti-Toxoplasmosis activity in vitro. They thus should be suitable for treatment of infection by intracellular parasites.

### Example 1

### Hydroxamic Acids as In Vitro Inhibitors of T. gondii

Materials and Methods: Unless otherwise noted, the following materials and methods were used in all of the following Examples.

Drugs—Trichostatin A, sodium valproate, 4-phenylbutyrate, and sodium butyrate were purchased from Sigma Chemical Company (St. Louis, Mo.). Scriptaid was purchased from BioMol International (Plymouth Meeting, Pa.). SAHA was the gift of Dr. Chris Reilly. TSA, SAHA and scriptaid were dissolved in DMSO as 10 mM stocks and stored at −20° C. They were diluted into culture medium immediately prior to use. The concentration of DMSO solvent in these experiments did not exceed 0.1%. Fresh stock solutions of sodium valproate, phenylbutyrate, and sodium butyrate were prepared in sterile phosphate-buffered saline and diluted into culture medium for each experiment. Control treated groups received an equal volume of DMSO or phosphate-buffered saline.

Parasite propagation—The RH strain of Toxoplasma gondii was grown in BM bovine macrophage cells or HS68 human foreskin fibroblast cells obtained from the American Type Culture Collection (Rockville, Md.). HS68 cells were grown in RPMI 1640 medium (Mediatech, Inc., Herndon, Va.) plus 10% fetal bovine serum (Atlanta Biologicals, Lawrenceville, Ga.), 1 mM sodium pyruvate and 100 U/ml each of penicillin and streptomycin. Parasites were harvested from infected cultures of HS68 cells growing in T-75 cm² flasks. After removing the culture medium, the cells were scraped into 5 ml of phosphate-buffered saline and a cell lysate obtained by passing through a 26-gauge needle. To purify the parasites, the cell lysate was filtered through a 3 micron (micrometer) filter.

Proliferation assays: HS68 cells were replica plated in 48-well dishes, and grown to confluence prior to infection with 5-10×10⁴ freshly purified T. gondii tachyzoites/well. After 2 hours, the media and non-infective tachyzoites were removed, and replaced with RPMI medium containing 2% fetal bovine serum plus 1 mM sodium pyruvate, penicillin (100 U), streptomycin (100 U) and test agent or a vehicle control. The proliferation assays were terminated 48-72 hours post-infection when the untreated cells were heavily infected. All of the culture media were removed from the wells, and the tachyzoites in the media were fixed by dilution (1:1) into phosphate-buffered saline containing 10% formaldehyde. The tachyzoites were collected by brief centrifugation (30 seconds, 80,000×g) at room temperature using a tabletop microfuge. The tachyzoites were resuspended in 100 ul of phosphate-buffered saline, and duplicate aliquots were counted using a hemocytometer. IC₅₀ values, the concentrations of inhibitors necessary to decrease the number of tachyzoites in the medium by 50%, were determined for each experiment using Prism GraphPad version 4.0 to fit the concentration-response data to a sigmoidal curve. The monolayer of HS68 cells in each well was fixed and stained for 5 minutes using a solution of 50% ethanol-5% formaldehyde-0.1% crystal violet in 0.85% sodium chloride, then rinsed with water, and air-dried. The minimum inhibitory concentration (MIC) of compounds that suppressed Toxoplasma-induced lysis of the HS68 cells was determined visually by counting the number of plaques/well in the monolayer.

Statistical analyses—Prism GraphPad version 4.0 was used to calculate experimental means, standard errors and to perform ANOVA. In the one-way ANOVA analyses of the hydroxamic acid concentration-response curves, Dunnett's t-test with P<0.05 was used to compare the effects of the histone deacetylase inhibitors with non-treated control samples.

Histone Deacetylase Inhibitors:

The purpose of this study was to compare the anti-Toxoplasma activity of the hydroxamic acid and carboxylate inhibitors shown in FIGS. 1A-1C.

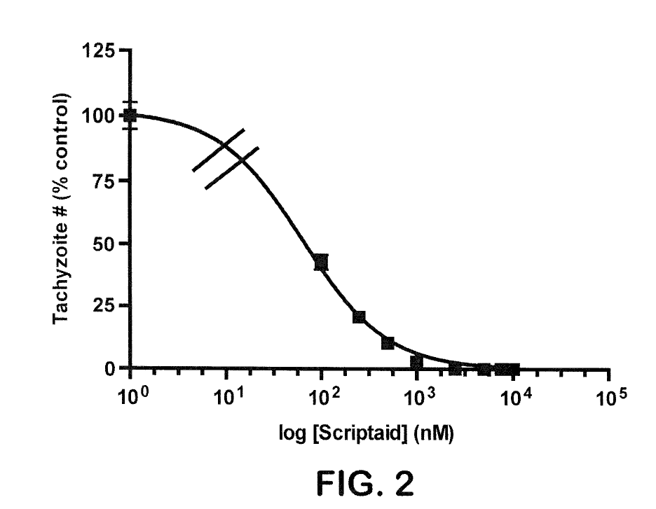

First, hydroxamic acid compounds were tested for their in vitro ability to inhibit growth of T. gondii Tachyzoites. SAHA, SBHA, scriptaid, and TSA were tested, and all were found to inhibit growth to some extent. However, scriptaid and SAHA were found to be potent anti-Toxoplasma compounds with low cytotoxicity. These agents reduced the number of tachyzoites released by infected cells with nanomolar IC₅₀ levels. Scriptaid was the most potent of all the inhibitors we tested. The average scriptaid IC₅₀ calculated in four independent experiments was 37 nM. A representative scriptaid concentration-Toxoplasma-response curve is presented in FIG. 2. Scriptaid was non-toxic to the HS68 cells at 10 uM, which was the highest concentration tested. SAHA also reduced Toxoplasma tachyzoite numbers (IC₅₀=83 nM) with no discernable toxicity to HS68 cells at the highest concentration tested, which was 10 uM.

Our results show that scriptaid and SAHA are far more potent inhibitors of growth of T. gondii tachyzoites than growth of tumor cells in vitro. In tests with a variety of human tumor cell lines growing in tissue culture, reductions in tumor cell numbers and induction of tumor cell apoptosis were achieved with 1-20 uM SAHA (see, for example, Komatsu et al., 2006; Sonnemann et al., 2006; Wilson et al., 2006; Zhang et al., 2005). Our findings show that for T. gondii tachyzoite growth the IC₅₀ is 83 nM SAHA and therefore, tachyzoites are ˜10-200 times more sensitive to SAHA than human tumor cells are. The differential sensitivity of T. gondii tachyzoites to scriptaid is even greater than that of SAHA. Scriptaid IC₅₀ levels between 3-60 uM (1-20 ug/ml) has been reported to reduce tumor cell proliferation in vitro (Keen et al., 2003; Su et al., 2000; Takai et al., 2006). In comparison, the IC₅₀ of 37 nM for T. gondii tachyzoites indicates that T. gondii tachyzoites are about 80-1500 times more sensitive to scriptaid than tumor cells are. These findings are clear support for use of scriptaid, SAHA, and other hydroxamic acids in therapeutic and prophylactic treatment of toxoplasmosis.

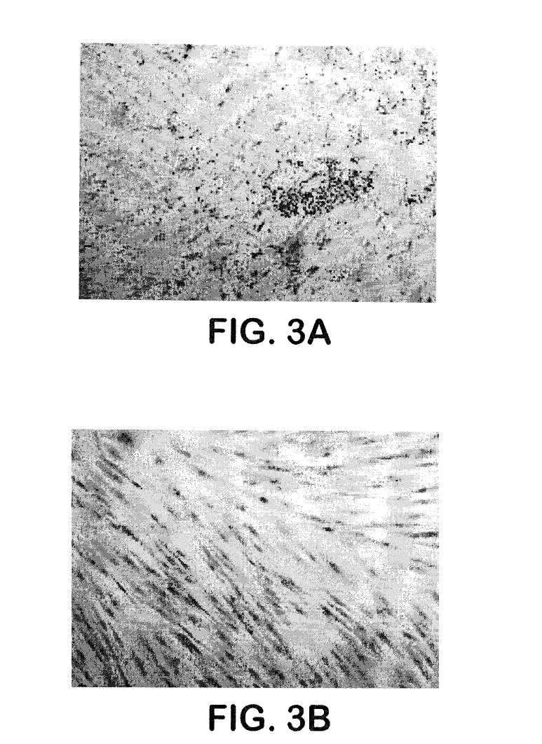

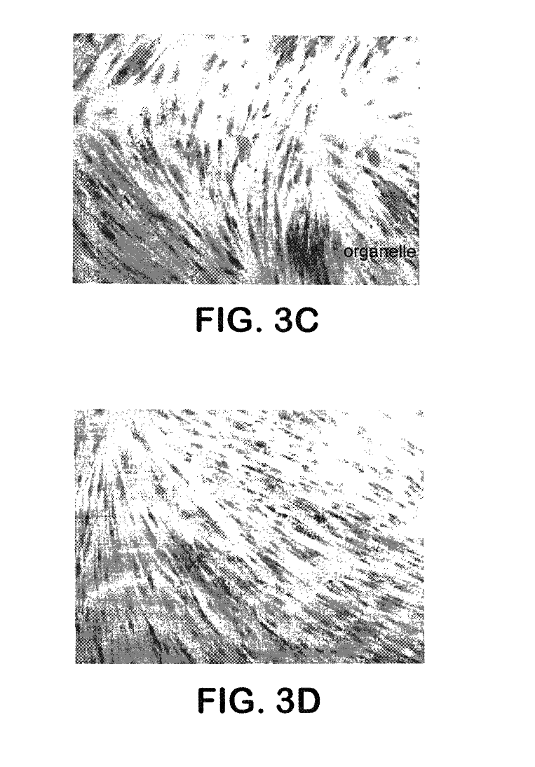

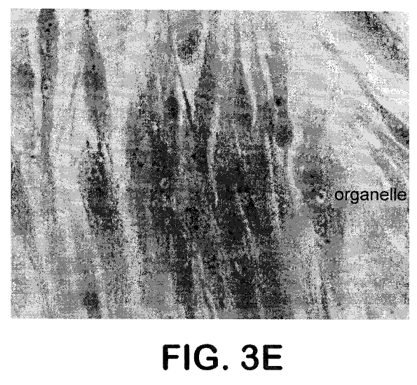

Photomicrographs comparing T. gondii-infected cells (FIG. 3A) or control HS68 cells after 48 hours in culture (FIG. 3B) show complete destruction of the cell monolayer by the tachyzoite infection. In contrast, when T. gondii-infected cells were treated with either SAHA (FIG. 3C) or scriptaid (FIG. 3D) for 48 hours, the monolayer was completely intact. These results suggest that the hydroxamic acid inhibitor treatments suppressed T. gondii infection and proliferation. Even 7 days later, no evidence of residual T. gondii appeared as plaques in the HS68 cell monolayer. No other agent tested was as effective as scriptaid or SAHA in these assays.

Careful examination of the HS68 monolayers that were treated with SAHA showed evidence of a persistent intracellular non-proliferating Toxoplasma zoites (FIG. 3E). There were no such organelles in the scriptaid treated cells, pointing to a possible difference in the anti-toxoplasma activity of scriptaid and SAHA.

Various other compounds that affect the growth of intracellular parasites can be immediately envisioned by those of skill in the art based on the representative species disclosed herein and the general compound presented in FIG. 1C and above as Formula I.

Low-dose Hydroxamic Acid Inhibitors Stimulate T. gondii Tachyzoites:

TSA, SAHA, and scriptaid had atypical effects in T. gondii infected HS68 cells in experiments where very low concentrations of hydroxamic acids were tested. As seen in FIG. 4, a biphasic response in the number of tachyzoites in the medium was observed. The stimulation of tachyzoite numbers with low TSA concentrations between 1 and 50 nM was reproducible. The dramatic decrease in tachyzoite numbers with TSA concentrations >100 nM statistically significant (p<0.05). Tachyzoites and plaque formation were reproducibly eliminated by 200 nM TSA under our experimental conditions.

More specifically, FIG. 4 is a composite of all the TSA, SAHA and scriptaid concentration-response curves for inhibition of T. gondii tachyzoites by TSA (solid circles), SAHA (solid squares), and scriptaid (open circles). Data are the mean+/−SEM of n=4-5 experiments performed in triplicate. For clarity, the standard error bars for 10 nM scriptaid (+/−1.9), which overlap those for TSA, were omitted. As can be seen from FIG. 4, a bimodal effect on growth of intracellular parasites is seen with increasing concentrations of inhibitors. Specifically, at amounts in the range reported in the literature as being effective at inhibiting the histone deacetylase activity of an HDAC in vitro (and thus function as inhibitors of parasite growth), certain compounds considered to be HDAC inhibitors in fact stimulate growth of intracellular parasites in vivo. It is not until significantly higher amounts of these compounds are administered, such as amounts that are effective at generating reactive oxygen species, that an inhibitory effect on growth is seen. To achieve in vivo effectiveness, one must administer a sufficient amount of a compound to generate DNA damage, for example by generation of reactive oxygen species. This surprising result does not follow from any previous work in this area, and the overlap between known HDAC inhibitors and compounds of the invention is merely coincidental. This coincidence is further evidenced by the fact that certain HDAC inhibitors do not function in vivo as inhibitors of growth of intracellular parasites.

We tested the effects of very low concentrations of scriptaid and SAHA on tachyzoite numbers, based upon the response we observed to low TSA. FIG. 4 shows there was also an increase in tachyzoite numbers in response to low concentrations of scriptaid and SAHA albeit less than to TSA. Stimulation of tachyzoite numbers by 1-10 nM scriptaid was observed in two of four experiments. However, in the wells showing increased tachyzoite numbers, the number of plaques that formed in the monolayers was not increased. We conclude that very low (nanomolar) concentrations of three potent hydroxamic acid histone deacetylase inhibitors, TSA, SAHA and scriptaid, do not decrease, and instead potentially stimulate, T. gondii tachyzoite proliferation and/or survival. This is finding has important implications for the mechanism of action of hydroxamic acid inhibitors in Toxoplasma biology. Specifically, the data suggest that concentrations of hydroxamic acid that inhibit human histone deacetylase in cell-free assays stimulate T. gondii proliferation.

TSA was also the most toxic to the HS68 cells of all the histone deacetylase inhibitors. The HS68 cell monolayer was destroyed by a 48-hour exposure to 1 micromolar TSA, and apoptotic cells were evident after exposure to 500 nM TSA. The cytotoxicity of TSA is well-documented, and precludes its therapeutic use Yoshida and Beppu, 1988).

Comparison of Histone Deacetylase Inhibitors on T. gondii IC₅₀ and MIC:

Table 1 reviews the anti-Toxoplasma activity of all the inhibitors we tested. The IC₅₀ and MIC values reported in Table 1 specifically apply to the 48-well plate assay as detailed in the Materials and Methods, above. Increasing or decreasing the number of tachyzoites used to infect the HS68 cells will shift the concentration-response curve to the right or left, respectively. Similarly, the MIC is dependent upon the tachyzoite numbers used in the infection, and higher inhibitor concentrations are needed to suppress plaque formation when more tachyzoites are used to infect the cells. At their MIC, the inhibitors were not cytotoxic to the HS68 cells. In general, we found that the MIC for anti-Toxoplasma activity of the hydroxamic acid and carboxylate histone deacetylase inhibitors was 3-6 times higher than the respective IC₅₀.

The least potent hydroxamic acid inhibitor was SBHA. SBHA showed good anti-Toxoplasma activity as measured by its ability to reduce tachyzoite numbers in the medium (IC₅₀), but unusually high concentrations of SBHA relative to its IC₅₀ were required for suppression of plaque formation.

TABLE 1
In vitro anti-Toxoplasma activity of Chemical Inhibitors

| | IC₅₀ (uM) | MIC (uM) | MIC |
|---|---|---|---|
| Agent | Tachyzoite Count | Plaque Suppression | IC₅₀ |
| Hydroxamic Acid | | | |
| Inhibitors | | | |
| Scriptaid | 0.039 ± 0.011 | 0.225 ± 0.025 | 5.8 |
| | n = 4 | n = 2 | |
| TSA | 0.041 ± 0.001 | 0.175 ± 0.025 | 4.3 |
| | n = 4 | n = 4 | |
| SAHA | 0.083 ± 0.04 | 0.400 ± 0.1 | 4.8 |
| | n = 5 | n = 4 | |
| SBHA | 0.213 ± 0.11 | 12.5 ± 5 | 58.7 |
| | n = 3 | n = 4 | |
| Carboxylic Acid | | | |
| Inhibitors | | | |
| Sodium butyrate | 1000 ± 455 | 4200 ± 800 | 4.2 |
| | n = 6 | n = 6 | |
| Valproic acid | 1600 ± 1116 | 8300 ± 1000 | 5.2 |
| | n = 3 | n = 3 | |
| 4-Phenylbutyrate | 5350 ± 2750 | 16,700 ± 3300 | 3.1 |
| | n = 2 | n = 3 | |

The IC₅₀ values in Table 1 were derived by averaging the IC₅₀ determined from n number of independent concentration-response curves. Data are mean+/−SEM in n independent experiments conducted in triplicate at each concentration.

Carboxylate Inhibitors of T. gondii Tachyzoites:

The carboxylates sodium valproate, sodium butyrate, and 4-phenylbutyrate were less potent than the hydroxamic acid inhibitors in reducing T. gondii tachyzoite numbers and plaque formation. The carboxylates are water-soluble and sufficiently high concentrations of all these compounds can be achieved in tissue culture to suppress T. gondii tachyzoite numbers and plaque formation in 48-72 h assays. However, when experiments were extended to 5-7 days, tachyzoites appeared in the medium, eventually leading to re-infection of the cell monolayer. We conclude that carboxylate inhibitors are not as efficacious as hydroxamic acids in the long-term suppression of T. gondii in vitro.

Discussion of Example 1 Data and Results:

Toxoplasma is a widespread and significant cause of disease in humans and domesticated cats. Livestock and wildlife, including feral cats, serve as important reservoirs of the disease. Antibiotics are effective treatments for the active stage of Toxoplasmosis marked by rapid tachyzoite proliferation, but there is no method to eradicate the T. gondii bradyzoites once tissue cysts have formed (Bonfioli and Orefice, 2005). The present invention shows that one suitable approach to the control of Toxoplasma is to utilize anti-cancer pharmacologic agents that target tachyzoites and prevent their conversion to bradyzoites.

The propagation of Toxoplasma gondii tachyzoites in human fibroblast cells in tissue culture was fully suppressed by the hydroxamic acid histone deacetylase inhibitors scriptaid and suberoylanilide hydroxamic acid (SAHA). The carboxylate histone deacetylase inhibitors, sodium butyrate, sodium valproate and 4-phenylbutyrate displayed anti-Toxoplasma activity at higher concentrations than the hydroxamic acid inhibitors.

The selective activation of apoptosis in tumor cells but not in normal cells is a hallmark of anti-cancer drug actions. Although changes in levels of expression of apopototic and anti-apoptotic proteins occur, the differential sensitivity of tumor cells to apoptosis has been linked to the defective G₂ checkpoint in cancer (reviewed in Gabrielli, 2006). TSA and SAHA cause DNA damage and the failure of the G₂ checkpoint to halt progression of cells with damaged DNA through the cell cycle triggers tumor cell apoptosis. The hydroxamic acid inhibitors TSA and SAHA, which are believed to be histone deacetylase inhibitors, produce intracellular reactive oxygen species and DNA damage through a process that is poorly understood (Martirosyan et al., 2006). Data provided herein suggests that the exquisite sensitivity of T. gondii tachyzoites to the hydroxamic acid histone deacetylase inhibitors might result from their high sensitivity to oxidative damage (Murray and Cohn, 1979) and the replication of T. gondii tachyzoites by an atypical eukaryotic cell cycle that lacks a G₂ phase (Khan et al., 2002; Radke et al., 2001).

In conclusion, the sensitivity of T. gondii to hydroxamic acid inhibitors is a new finding with direct application to the prophylaxis of Toxoplasmosis and development of new treatments for Toxoplasmosis and other intracellular parasites in humans and animals.

In view of the above disclosure, it should be apparent that, in various embodiments, the invention provides methods of prophylaxis and therapy of Toxoplasma gondii infections. Among the various uses are: prophylaxis for Toxoplasmosis in organ transplantation, treatment of ocular Toxoplasmosis, treatment of systemic Toxoplasmosis in immune compromised patients (e.g., HIV+ persons, persons receiving immune suppressant drugs for chronic immune disorders, cancer patients, the elderly, malnourished patients), treatment of patients with schizophrenia, manic-depressive disorder, hallucinations that are unresponsive to standard neuropharmacologic agents, and treatment of newborn children of mothers who acquired infections of T. gondii during pregnancy. In the veterinary field, treatment can be for treatment of Toxoplasmosis in felines (e.g., house pets, zoos, etc.), and treatment of Toxoplasmosis in wildlife housed in zoos. These embodiments flow from the data presented herein relating to in vitro anti-Toxoplasma activity of SAHA and scriptaid, and the in vivo anti-Toxoplasma activity of SAHA described herein and in the priority documents. The methods of treatment also are supported by the information in Example 4, below, which shows the in vitro activity of compounds of the invention in oxygen-free radical production in human tumor cells, an established mode of anti-tumor action. That Example also shows a link between NSC3952, reactive oxygen species, and cell differentiation and apoptosis in MCF-7 human mammary tumor cells.

It should also be apparent from the above disclosure that, in embodiments, the invention provides for the use of scriptaid, SAHA, and other compounds of the invention in the prophylaxis and therapy of Cryptosporidiosis caused by Cryptosporidium parvum and Cryptosporidium hominus. Among the various uses are: treatment of active C. parvum infections acquired by water-borne transmission, and prophylaxis of C. parvum infections in exposed populations due to water contamination, either by accidental or intentional (bioterrorism) contamination of drinking water supply, swimming pools, spas, and water parks. These uses flow from the extension of the data presented herein to activity on other highly related species and proteins.

For, example, it flows from, among other things, the in vitro anti-C. parvum activity of SAHA. Monolayer cultures of HCT-8 human colon cells growing in 24-well plastic tissue culture dishes were infected with C. parvum oocysts for 3 hours. The non-infective oocysts were removed, and the infected cells were treated for 48 hours with control medium, medium plus 0.1% DMSO (solvent), 70 ng/ml apicidin (an active anti-C. parvum agent), or 2.5 ug/ml SAHA. The cells were harvested, and DNA was extracted. The DNA was analyzed for the production of C. parvum oocysts from the infected cells using real-time PCR and DNA primers that specifically amplify the C. parvum oocysts wall protein (COWP). The results show that SAHA statistically significantly reduced the number of C. parvum oocysts released from the infected cells (one-way ANOVA, Dunnett's t-test, P<0.01). The results are shown in Table 2.

TABLE 2
Real-Time PCR Analysis of Cryptosporidium parvum Oocyst
Production in infected HCT-8 Human Colon Tumor Cells

| | number of oocysts/ml | | |
|---|---|---|---|
| Treatment | (mean ± S.D. | Statistical | % Reduction in |
| Group | n = 6 | Significance | oocysts numbers |
| 0.1% DMSO | 6661 ± 6371 | — | — |
| Apicidin | 517.7 ± 417.1 | P < 0.05 | 92.2 |
| (70 ng/ml) | | | |
| SAHA | 252.8 ± 280 | P < 0.05 | 96.2 |
| (2.5 ug/ml) | | | |

Based on this data, it can be concluded that apicidin and SAHA exhibit anti-cryptosporidial activity in vitro.

In addition, the invention provides, in embodiments, for use of scriptaid, SAHA, and other compounds of the invention as treatments for infectious diseases caused by Apicomplexa family members, in particular those closely related to Toxoplasma (e.g., Eimera tenella, Plasmodium falciparum, Sarcocystis neurone, and Neospora caninum). Treatments can be, but are hot necessarily limited to: treatment of P. falciparum, such as by treatment of all malarial strains in humans, including chloroquine-resistant malaria, quinidine-resistant malaria, artemisinin-resistant malaria, and multi-drug resistant malarial strains. It can also include chemoprevention for P. falciparum for travelers to endemic malarial areas and military personnel stationed in endemic malarial areas.

Further, the invention provides, in embodiment, prevention and therapy for infections with E. tenella, such as prevention of sub-clinical and clinical coccidiosis in chickens and turkeys. Furthermore, prophylaxis and therapy for Sarcocystis neurone is provided, which can, in embodiments, take the form of prevention and/or treatment of equine protozoal myeloencephalitis caused by Sarcocystis neurone, or in embodiments, Neospora hughesi.

In some embodiments, treatment for infections of Neospora caninum are also provided. These embodiments can include prevention of abortion due to primary or reactivated N. caninum infection in cattle.

Many of the embodiments of the methods of treating follow from the relatedness of many intracellular parasites. Comparative genomic evidence exists for a significant similarity of the gene/protein sequences of the members of Apicomplexa species P. falciparum, C. parvum, C. hominus, and T. gondii for which complete genome sequences are now available. Some data relating to these organisms is provided in Table 3.

TABLE 3
Summary of HDAC Sequence Homologies

| | Gene | Sequence | Sequence | Gaps in |
|---|---|---|---|---|
| | Identifier | Identity | Positives | Alignment |
| E. tenella vs. | CD344993 vs. | 90% identify | 95% positives | No gaps, 118 |
| T. gondii | AAY53803.1 | | | amino acid |
| (hdac | BLASTx | | | segment |
| proteins) | | | | |
| N. caninum | CF941786 vs. | 87% identity | 93% positives | 2% gaps, 49 |
| vs. T. gondii | AAY53803.1 | | | amino acid |
| (hdac | BLASTx | | | segment |
| proteins) | | | | |
| C. parvum vs. | XP_62348.1, | 77% identity | 89% positives | 0% gaps, 383 |
| T. gondii | CAD98610.1, | | | amino acid |
| (hdac | AAG21919.1, | | | segment |
| proteins) | vs. | | | |
| | AAY53803.1 | | | |
| | BLASTp | | | |
| C. hominus | XP_667698.1 | 77% | 89% | 0% gaps, 383 |
| vs. T. gondii | v. | | | amino acid |
| | AAY53803.1 | | | segment |
| | BLASTp | | | |
| P. falciparum | CAD51938.1, | 74% identity | 85% positive | 0% gaps, 443 |
| vs. T. gondii | NP_704795.1, | | | amino acid |
| | AAD22407.1 | | | segment |
| | v. | | | |
| | AAY53803.1 | | | |
| | BLASTp | | | |

Full length T. gondii hdac = 451 amino acids

### Example 2

### In Vivo Anti-Toxoplasmosis Activity of Hyrdroxamic Acid Inhibitors

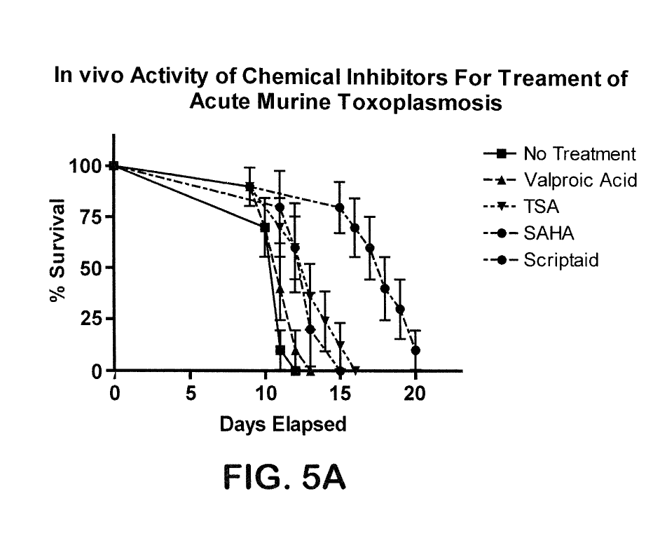

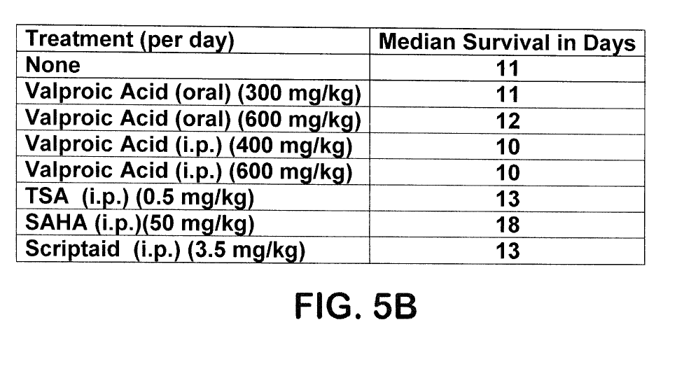

To extend the in vitro data developed and reported above, in vivo studies were carried out to determine the effectiveness of compounds of the present invention in treating intracellular parasites. Specifically, female CD-1 mice (20 g) were inoculated in the sub-scapular region with 5,000 T. gondii tachyzoites on Day 0. TSA, SAHA, and scriptaid were dissolved in DMSO and administered in a DMSO-saline solution once daily by i.p. injection beginning on day −1. The doses used in this study were: TSA (0.5 mg/kg), SAHA (50 mg/kg) and Scriptaid (3.5 mg/kg). The doses of TSA and SAHA are near maximal tolerated doses in this species. The dose of scriptaid was not optimized in this experiment. In this study, valproic acid was administered orally in the drinking water at a dose of 300 mg/kg. Survival was monitored twice daily beginning on day 9 of the study. The results are depicted graphically in FIG. 5A and in tabular form in FIG. 5B.

As can be seen from the data in FIG. 5, the hydroxamic acid inhibitors (TSA, SAHA, and scriptaid) showed in vivo efficacy in the control of acute toxoplasmosis in mice. In contrast, valproic acid, a carboxylic acid inhibitor, was ineffective in the control of acute toxoplasmosis in mice. The data shown in FIG. 5 are results of a daily oral dose (300 mg/kg) of valproic acid. In other experiments, valproic acid was also ineffective at an oral dose of 600 mg/kg (median survival 12 days); valproic acid was ineffective after i.p. administration at doses of 400 mg/kg and 600 mg/kg divided into 2 daily doses (median survival 10 days). All of these results are summarized in the Table of FIG. 5B.

### Example 3

### Anti-Cancer Activity of a Compound of the Invention by Generation of Reactive Oxygen Species

This study was preformed to show that other compounds according to the invention are suitable for use as inhibitors of intracellular parasites. While the data shows such an activity is present, it also shows that the compound, NSC3852 (5-nitroso-8-quinolinol) links reactive oxygen species to cell differentiation and apoptosis in MCF-7 human mammary tumor cells.

More specifically, it was found that NSC3852 has cell differentiation and anti-proliferative activity in human breast cancer cells in tissue culture and anti-tumor activity in mice bearing P388 and L1210 leukemic cells. We investigated the mechanism of NSC3852 action in MCF-7 human breast cancer cells using electron spin resonance (ESR). Reactive oxygen species (ROS) were detected in MCF-7 cell suspensions incubated with NSC3852 using the spin trap 5,5-dimethyl-1-pyrroline-N-oxide (DMPO). Formation of the DMPO-OH adduct was quenched by the addition of superoxide dismutase but not by catalase, and we concluded that superoxide was generated in the NSC3852-treated cells. The flavoprotein inhibitor diphenylene iodonium suppressed ROS production, providing evidence for the involvement of a flavin-dependent enzyme system in the ROS response to NSC3852. A biologically significant oxidative response to NSC3852 occurred in MCF-7 cells. An early marker of oxidative stress was a decrease in the [glutathione]/[glutathione disulfide] ratio 1 h after NSC3852 addition. Oxidative DNA damage, marked by the presence of 8-oxoguanine, and DNA-strand breakage occurred in cells exposed to NSC3852 for 24 h. Apoptosis peaked 48 h after exposure to NSC3852. Pretreatment with the glutathione precursor N-acetyl-L-cysteine (NAC) prevented DNA-strand breakage and apoptosis. Pretreatment with NAC also reversed NSC3852 decreases in E2F1, Myc, and phosphorylated retinoblastoma protein, indicative of redox-sensitive pathway(s) in MCF-7 cells during G1 phase of the cell cycle. We conclude that ROS formation is involved in the apoptotic and cell differentiation responses to NSC3852 in MCF-7 cells.

Differentiation agents are promising experimental antitumor agents that modify epigenetic pathways in tumor cells. Differentiation agents cause growth arrest and expression of proteins typical of the normal cell phenotype in cancer cells, but these are believed to be transient effects. The ability to trigger apoptosis in tumor cells is critical to the antitumor activity of differentiation agents; the mechanisms leading to tumor apoptosis vary with the individual differentiation agent and tissue type. We investigated the mechanism of the cell death and differentiation in human breast tumor cells exposed to a new antitumor agent, NSC3852 (5-nitroso-8-quinolinol).

A large number of inhibitors of class I/II histone deacetylases are now characterized, and we know that a variety of chemical structures possess histone deacetylase inhibitory activity. The main classes of histone deacetylase inhibitor(s) are 1) short-chain fatty acids, 2) hydroxamic acids, 3) cyclic tetrapeptides with and without amino-epoxy-oxodecanoic acid residues, and 4) benzamides. The hydroxamic acids, represented by suberoylanilide hydroxamic acid (SAHA) and trichostatin A (TSA), are among the most potent. NSC3852 is less potent than SAHA, but has many similar effects in MCF-7 cells. Both cause growth arrest and accumulation of cells in G0 phase of the cell cycle, as well as morphologic changes, such as formation of cytoplasmic lipid droplets and an enlargement of the cytoplasm. All of these inhibitors now appear to bind the zinc ion at the enzyme active site. NSC3852 lacks the hydroxamic acid moiety that is responsible for SAHA binding to Zn²⁺ in the histone deacetylase active site. NSC3852 harbors a different Zn²⁺ chelation motif, 8-quinolinol. For this study, we postulated that N5C3852 might exhibit novel actions in MCF-7 cells, because the quinoline ring and the nitroso substituent place N5C3852 in a chemically unique category of inhibitors. Our studies revealed that NSC3852, SAHA, and TSA stimulated ROS formation in MCF-7 cells and that N5C3852-induced oxidative stress contributed to apoptosis and differentiation in MCF-7 cells.

Materials and Methods: The following materials and methods were used in this Example unless otherwise noted.

Tissue Culture: The MCF-7 human breast cancer cells (passage 40-55) were maintained in Dulbecco's modified Eagle's medium (DMEM) (BioWhittaker, Walkersville, Md.) supplemented with 10% heat-inactivated fetal bovine serum (FBS), 2 mM glutamine, and 40 ug/ml gentamicin at 37° C. in a humidified atmosphere of 6% CO₂ and 94% air. Cells were passaged every 4 to 5 days. Experiments were carried out in DMEM supplemented with 5% FBS. Cells were counted using a hemocytometer and 0.02% trypan blue to assess cell viability.

Chemicals: NSC3852 and NSC2039 (8-quinolinol) were kindly provided by Dr. Robert Schultz (Drug Synthesis and Chemistry Branch, Developmental Therapeutics Program, Division of Cancer Treatment and Diagnosis, National Cancer Institute, Bethesda, Md.). In this study, we used concentrations of NSC3852 and NSC2039 that inhibited proliferation of MCF-7 cells by 50% in a standard MTS cell proliferation assay. TSA was from Upstate Biotechnology (Lake Placid, N.Y.). SAHA and suberic bishydroxamate (SBHA) were gifts from Dr. Q. Zhou (Johns Hopkins University, Baltimore, Md.). N-Acetyl-L-cysteine (NAG) and N^{G}-nitro-L-arginine methyl ester were from Sigma-Aldrich (St. Louis, Mo.). Dihydroethidium and 2′,7′-dihydrodichlorofluorescein diacetate were purchased from Molecular Probes (Eugene, Oreg.).

Western Blot Analyses: Cells (2×10⁶/60 mm2) were treated 12 h after plating. Cells were harvested by scraping in 100° C. lysis buffer (1% SDS and 10 mM Tris, pH 7.4) and heated (100° C., 5 mm), and extracts were prepared by centrifugation in a microcentriftige (4° C., 5 mm). Protein concentrations were determined in aliquots of these extracts using the bicinchoninic acid assay (Pierce, Rockford, Ill.). Dithiothreitol (1 mM) and protease inhibitors were added (phenylmethylsulfonyl fluoride (1 mM), aprotinin (1 ug/ml), and leupeptin (1 ug/ml)) to the extracts. Protein samples (60-70 ug) were electrophoresed on 10% polyacrylamide gels and transferred to polyvinylidene difluoride membranes (Invitrogen, Carlsbad, Calif.). Membrane blocking and incubations with primary and secondary antibodies were performed according to standard procedures. Chemiluminescent signals were recorded on X-ray film and quantified using Fluor Chem (Alpha Innotech, San Leandro, Calif.) spot densitometry program with automatic background subtraction.

Free Radical Measurements: All ESR measurements were conducted using a Bruker EMIR spectrometer (Bruker Instruments Inc., Billerica, Mass.) and a flat-cell assembly. Hyperfine couplings were measured (to 0.1 G) directly from magnetic field separation using potassium tetraperoxochromate (K₃CrO₈) and 1,1-diphenyl-2-picrylhydrazyl as reference standards. The relative radical concentration was estimated by multiplying half of the peak height by (ΔHₚₚ)², where (ΔHₚₚ) represents peak-to-peak width. The Acquisit program was used for data acquisitions and analyses. MCF-7 cells (2.0×10⁶ cells/60-mm² dish) were harvested 48 h after plating by trypsinization and collected at room temperature by centnfugation (225 g, 5 min). Cells were washed once with ice-cold PBS and resuspended in ice-cold PBS at 2×10⁶ cells/ml. Radical production was measured in the presence of the spin trap 5,5-dimethyl-1-pyrroline-N-oxide (DMPO; Aldrich Chemical Co., Milwaukee, Wis.). DMPO (200 mM) and 1×10⁶ cells test agents were mixed in 1.0 ml of PBS, incubated at 37° C. for 5 mm, and then transferred to a flat cell for ESR measurements.

Intracellular Reactive Oxygen Species Detection: Cells were plated (2×10⁵/35-mm² dish) in 3 ml of DMEM/5% FBS culture medium. After 12 h, cells were exposed to solvent control or 10 uM NSC3852. After the treatment, cells were exposed to 5 uM 2′,7′-dihydrodichlorofluorescein diacetate or dihydroethidium for 30 min. Cells were washed twice with PBS, and fluorescence was analyzed (10,000 events) using a FACScalibur flow cytometry (Becton Dickinson, San Jose, Calif.). Alternatively, cells plated onto sterile glass coverslips were fixed in 10% formaldehyde and examined using fluorescent confocal microscopy (Zeiss LSM 510).

Colorimetric Determination of Reduced and Oxidized Glutathione: The GSH and GSSG concentrations in MCF-7 cells (3×10⁶ cell/60 mm² dish) were measured enzymatically using the GSHI GSSG-412 kit (Oxis Research, Portland, Oreg.). GSSG and GSH standards were prepared in 5% metaphosphoric acid. Cell samples were prepared in 5% metaphosphoric acid with or without 1-methyl-2-vinyl-pyridium trifluoromethane sulfonate, a GSH-specific scavenger. Ellman's reagent (5′,5-dithiobis-2-nitrobenzoic acid) reacts with GSH to form a product with an absorption maximum at 412 nm. GSSG was determined using glutathione reductase to reduce GSSG to GSH followed by reaction with Ellman's reagent.

Comet Analysis: The comet assay is a single-cell gel electrophoresis method for measuring DNA damage. MCF-7 cells (2×10⁵/35-mm² dish) were treated 12 h after plating. Twenty-four hours later, the cells were harvested and resuspended (1.5×10⁵ cells/ml) in ice-cold PBS. The PBS cell suspension (50 ul) was mixed with 500 ul of 42° C. low-melting point agarose, spread evenly (75 ul) onto a Comet Slide (Trevigen, Gaithersburg, Md.), and allowed to harden. The slides were then immersed in ice-cold lysis solution (Trevigen) for 45 mm to lyse the cells and then transferred to freshly prepared alkali solution (300 mM NaOH and 1 mM EDTA, pH 8.0) for 45 mM. at room temperature to denature the DNA. Electrophoresis was performed at 4° C. Slides were air-dried overnight at room temperature and then stained with SYBR Green (Trevigen). Comets were visualized by fluorescence microscopy at 630× magnification with the aid of an antifade solution. Comet images were analyzed using the LAI Automated Comet Assay Analysis System (Loats Associates, Inc., Westminster, Md.). DNA damage was quantified in 80 comets per treatment/experiment using the tail moment [Tail moment [(% DNA) (distance traveled)]].

Detection of Oxidative Damage to DNA: Oxidative damage to DNA was determined using the OxyDNA assay (Biotrin, Dublin, Ireland). MCF-7 cells were plated onto sterile glass coverslips in 35-mm² tissue culture dishes and treated with N5C3852 12 h later. After 24 h, cells were fixed and permeabilized with 99% methanol. Nonspecific binding sites were blocked using blocking solution (1 h, room temperature). After washing twice, the cells were incubated in the dark with fluorescein isothiocyanate-conjugated antibody (1 h, room temperature) to identify 8-oxoguanine containing DNA. Cell images were captured using confocal microscopy.

Cell Death Enzyme-Linked Immunosorbent Assay: We quantified cytoplasmic nucleosomes using the Cell Death Detection ELISAPLUS kit (Roche Molecular Biochemicals, Indianapolis, Ind.) in MCF-7 cells plated in triplicate into 96-well plates. The cytoplasmic fractions from attached cells in each well were assayed in duplicate for the presence of nucleosomes according to the directions from the suppliers. The apoptotic response was measured by the nucleosome-enrichment fraction calculated as the ratio of the absorbance (405 nm) of drug-treated cultures/solvent-exposed cultures.

Statistics: Sigma plot software (SPSS Inc., Chicago, Ill.), version 5.0, and Prism (GraphPad Software, Inc., San Diego, Calif.), version 3.0, were used for statistical analysis. Statistically significant differences (P<0.05) were determined using Student's t test or a one-way ANOVA and Tukey's t test (P<0.01).

Results:

Free Radical Generation in MCF-7 Cells Mediated by NSC3852: The structures of NSC3852 and NSC 2039 are known in the art. NSC2039 is a control in our experiments, because it lacks the cell differentiation and histone deacetylase inhibitory activities of NSC3852. We used ESR to examine ROS formation in MCF-7 cell suspensions. The ESR signals obtained with MCF-7 cell suspensions containing 10 uM NSC3852 or 8 uM NSC2039 were determined (data not shown). In cells exposed to NSC3852, a 1:2:2:1 quartet signal (with a_{H}=a_{N}=14.9 G, where a_{H} and a_{N} denote hyperfine splitting of the alpha-hydrogen and the nitroxyl nitrogen, respectively), indicative of the DMPO-OH adduct, was observed. In MCF-7 cells treated with NSC2039, no signal was produced demonstrating that the nitroso substituent was required for the generation of ROS.

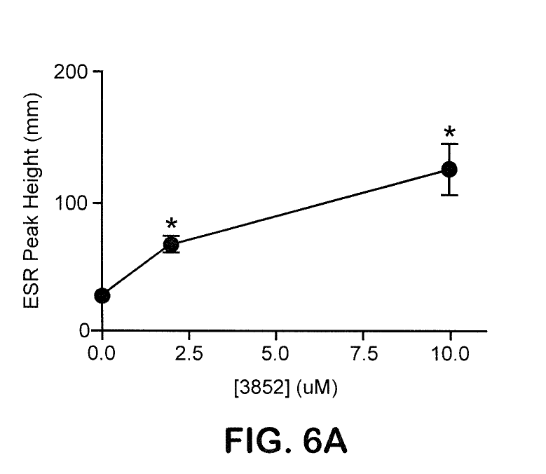

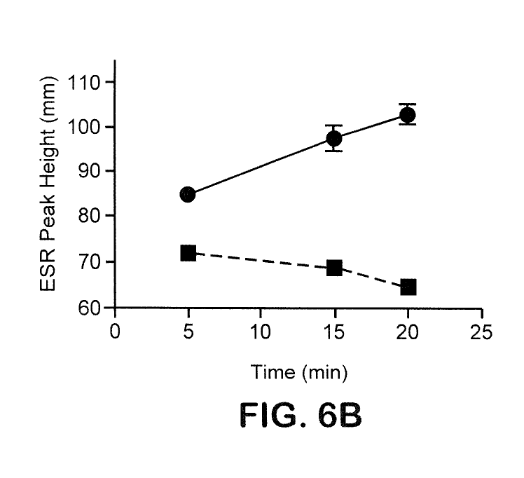

FIG. 6A shows the concentration-response relationship for ROS production in MCF-7 cells exposed to NSC3852. Statistically significant (P<0.01) ESR peak-height differences from controls are indicated (*). Panel B shows a time course of ROS production in MCF-7 cells+N5C3852. ESR spectra of 1×10⁶ MCF-7 cells+NSC3852+200 mM DMPO in PBS. FIG. 6A shows that there is a concentration-response relationship between the ESR-peak height in MCF-7 cells exposed to dimethyl sulfoxide solvent alone, 2 uM N5C3852, and 10 uM NSC3852. Statistically significant DMPO-OH peaks were generated at both the 2 and 10 uM NSC3852 concentrations. FIG. 6B compares the time course of the signal intensity of the DMPO-OH peak in cells treated with 2 or 10 uM NSC3852. The MCF-7 ROS response to 2 uM NSC3852 decayed more rapidly than response to 10 uM. In cells treated with 10 uM NSC3852, ROS production was stable for as long as we measured the reaction (25 min). No ROS were detected when NSC3852 or NSC2039 was diluted into PBS without MCF-7 cells, suggesting that cellular metabolism is involved in the ROS response to NSC3852.

To probe the source of the DMPO-OH ESR signal, either superoxide dismutase (SOD) or catalase was added to cell suspensions. Catalase, a scavenger of H₂O₂, had no effect upon the signal (data not shown). Therefore, the source of the DMPO-OH signal was unlikely to be OH derived from H₂O₂. An alternative route for the formation of DMPO-OH is through the trapping of superoxide to form the unstable adduct, DMPO-OOH that quickly decomposes to DMPO-OH. When SOD was added to the cell suspensions, the ESR signal was quenched (data not shown), consistent with data showing that, when SOD clears the superoxide very quickly, trapping of the unstable DMPO-OOH intermediate is suppressed. These results support our conclusion that the observed ESR spectra were produced by the decomposition of the DMPO-OOH signal trapped from superoxide.

The two major cellular sources for production of superoxide are flavoproteins in NADPH oxidase complexes and in the mitochondrial electron transport chain. Diphenyliodonium is a non-selective flavoprotein inhibitor affecting all of the flavin-dependent enzymes. Because DMPO can enter cells, the ESR spectra show both intracellular and extracellular ROS.

ESR spectra showing suppression of the DMPO-OH signal upon the addition of the enzyme SOD to the cell suspension were also obtained (data not shown). They suggested that at least half of the ESR signal was produced extracellularly. The nonphagocytic form of membrane NADPH oxidase, a flavin-dependent enzyme, is the most likely source of extracellular superoxide.

We also demonstrated intracellular superoxide production in MCF-7 cells in response to 10 uM NSC3852 using the fluorescent probes dihydroethidium and 2′-7′-dihydrodichlorofluorescein diacetate (data not shown). The identity and relative contribution of the enzymes responsible for the intracellular ROS response of MCF-7 cells to NSC3852 are not fully established; however, the complete inhibition of the ESR signals with DPI strongly implicated flavin-dependent enzymes, such as NADPH oxidase, the mitochondrial Complex I, nitric-oxide synthase, aromatase, and other cytochrome P450-dependent enzymes present in breast cancer. Using 1 mM N^{G}-nitro-L-arginine methyl ester to inhibit nitric-oxide synthase in MCF-7 cells, we showed a 29.5% (P<0.01) reduction in the DMPO-OH ESR signal intensity, indicating that nitric-oxide synthase is important to the mechanism of action of NSC 3852.

Augmentation of the NSC3852-generated DMPO-OH ESR signal by rotenone (data not shown is evidence for a role of mitochondrial Complex I in the intracellular ROS response to NSC3852. When rotenone binds Complex I, electron transport to ubiquinone is interrupted, resulting in superoxide formation. Alternatively, there is evidence in brain microglia that rotenone can directly stimulate NADPH oxidase activity, thus enhancing the DMPO-OH signal intensity. Thus, our data support a model in which NSC3852 stimulates ROS production through a variety of cellular-dependent pathways.

Regulation of G1 Proteins by NSC3852 and Reversal by N-Acetyl-L-cysteine. Cell-cycle arrest in G1 is a prerequisite for differentiation of human breast tumor cells in culture: Initial responses to differentiation stimuli include a shift in the expression of cell-cycle regulatory proteins toward their status in early G1 phase of the cell cycle, specifically hypophosphorylated retinoblastoma protein (Rb), and decreases in E2F1 and Myc protein. Earlier work showed that MCF-7 cells treated with NSC3852 accumulated in G0 after 48 h but that MCF-7 cells exposed to the related analog, NSC2039, did not. Based upon the differential ability of NSC3852 and NSC2039 to generate ROS and to cause differentiation in MCF-7 cells, we performed Western blot analyses of Rb, E2F1, and Myc (data not shown). NSC3852 caused a time-dependent accumulation of hypo-phosphorylated Rb and loss of phosphorylated Rb between 12 and 48 h. There were no detectable differences in Rb between control and NSC3852-treated cells at 8 h or earlier. Statistically significant decreases in phosphorylated Rb and E2F1 protein levels were seen at 12 and 24 h. After 24 h, Myc protein levels had decreased significantly, and by 48 h, Myc was undetectable in the Western blots. The time-dependent shift in the profile of Rb, E2F1, and Myc expression is consistent with the progression of NSC3852-treated cells into G0.

NSC3852 Has an Effect on [GSH]/[GSSG] Ratio in MCF-7 Cells: The major redox couple in mammalian cells is GSH-GSSG, and decreases in the intracellular [GSH}/[GSSG] ratio are a biological indicator of oxidative stress. NSC3852 stimulated superoxide levels within 15 min sufficiently to raise intracellular [GSSGI and thereby decrease the [GSH]/[GSSG] ratio. After 1 h, NSC3852 decreased the [GSH]/[GSSG] by 20% compared with control cells (P<0.05). By 6 h, the [GSH]/[GSSGI ratios in control and treated cells were statistically indistinguishable, suggesting that NSC3852 mediated a transient oxidative shift in the cellular redox potential.

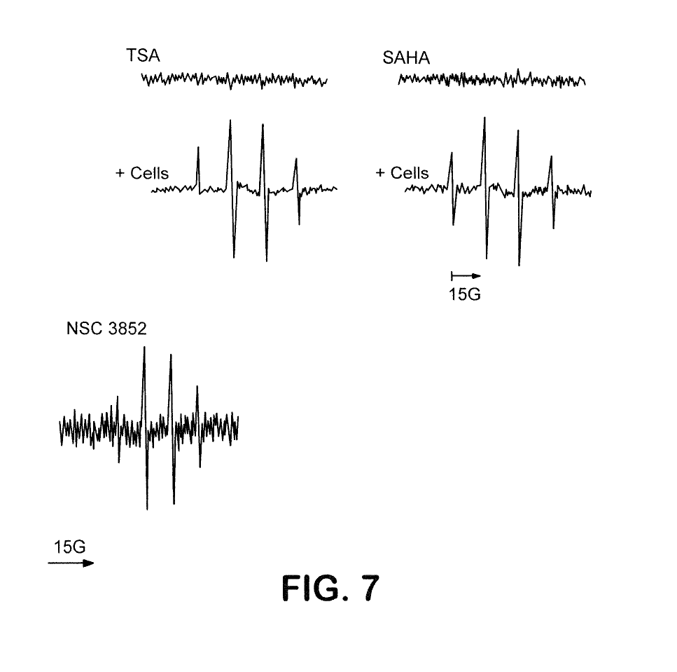

Free Radical Generation in MCF-7 Cells by Histone Deacetylase Inhibitors: NSC3852 is not unique among tumor differentiation agents in eliciting ROS. Three additional HDI (TSA, SAHA, and SBHA) elicited rapid and robust ESR signals indicative of ROS production in MCF-7 cells. The relative peak heights of the ESR signals (mean±range, n=2) were measured in parallel aliquots of 1×10⁶ MCF-7 cells treated with 200 nM TSA (99±1 mm), 200 nM SAHA (98 mm), 10 uM 3852 (109±3 mm), or 2 uM 3852 (70±4 mm) for 5 min at 37° C. We conclude that the magnitude of the ROS signals produced by these concentrations of HDI is roughly equivalent (see FIG. 7). Previous work using a ROS-sensitive fluorescence dye showed that SAHA and another HDI, MS-275, increased ROS production in human leukemia cells. This report is the first to demonstrate ROS generation in response to HDI using ESR.

DNA Damage and Apoptotic Effects of NSC3852 in MCF-7 Cells: ROS and oxidative stress produce DNA damage that can trigger apoptosis. We performed comet assays to test for pNA damage by NSC3852 and measured the extent of damage as the increase in the comet tail moment. NSC3852-induced DNA damage was detected after 12 h and was maximal at 24 h. DNA damage was undetectable after 96 h (data not shown), at which time an apoptotic response might have eliminated cells with damaged DNA. Therefore, after treatment with NSC3852, the cells behaved as if they were exposed to an oxidative stress that was either self-limiting or attenuated by compensatory cell-survival pathways. Three lines of evidence support the role of ROS in the DNA-damage response. First, pretreatment with 5 mM NAC 1 h before NSC3852 prevented DNA damage as measured at the 24-h time point of peak damage (data not shown). Second, NSC2039 did not induce DNA damage in MCF-7 cells (data not shown). Third, we used immunohistochemistry to detect 8-oxoguanine, a DNA adduct formed during free radical damage to DNA. All cells in the NSC3852-treated group contained nuclear 8-oxoguanine after 24 h, whereas control cells stained very weakly for this adduct (data not shown). Apoptosis occurred in response to NSC3852, and a 1-h pretreatment with NAC blocked this response (data not shown). The peak NSC3852 DNA-damage response occurred at 24 h (data not shown) and preceded the peak in NSC3852-induced apoptosis (48 h). This temporal relationship fits a model where NSC3852-induced formation of 8-oxoguanine DNA adducts leads to DNA-strand breakage and apoptotic cell death.

Discussion of the Results of this Example:

NSC3852 is known as a breast cancer differentiation agent with histone deacetylase activity. The purpose of this work was to understand the mechanistic basis for its pleiotropic actions in human breast cancer cells that are of potential significance in the treatment of cancer. One important finding was the enrichment of Rb in its hypophosphorylated state in NSC 3852-treated cells. Hypophosphorylated Rb is the active tumor suppressor state of Rb and is a marker of cells arrested in G1, cell differentiation, and cell senescence. We also showed that NSC3852 is a redox-active compound that stimulates superoxide production and a transient rise in intracellular redox potential. Our studies demonstrated that ROS production is important to the mechanism of action of NSC3852. ROS generation is dependent upon the interaction of NSC3852 with the cells and occurs both intracellularly and extracellularly. We propose that ROS production has dual actions in MCF-7 cells stimulating apoptosis through a well established mechanism of oxidative DNA damage and promoting cell differentiation through its actions on Rb protein. The basis for our hypothesis that NSC3852 acts on Rb phosphorylation status by a redox mechanism is the reversal of NSC3852 activity by N-acetyl-L-cysteine pretreatment and the failure of NSC2039, a non-ROS-generating analog, to change Rb status. The temporal series of events following treatment with NSC3 852 suggest that a ROS-initiated cascade of events leads to hypophosphorylated Rb, but our data do not exclude a direct action of ROS upon Rb. Other investigators have also concluded that cellular redox status regulates the phosphorylation state of Rb.

The work of others suggests that protein phosphatase 2A, the enzyme responsible for the dephosphorylation of Rb, responds to cellular redox status. These investigations showed that, as the intracellular redox potential rises in response to oxidative stress, phosphatase 2A activity is stimulated, increasing hypophosphorylated Rb. The idea that Rb acts as a thiol-dependent nanotransducer of intracellular redox state and cell-cycle progression/differentiation status is intriguing and is the foundation of a model of redox control of cell proliferation. However, there is substantial support for the opposing hypothesis that oxidative stress is mitogenic and antioxidants suppress proliferation. All thiol residues on proteins are redox-sensitive and theoretically transduce redox signals rapidly and reversibly via changes in protein structure. A broader examination of redox signaling in cell-cycle regulatory pathways is one approach to resolving this paradox.

Our model describing a dual role for ROS in the cell differentiation and apoptotic responses to NSC3852 in MCF-7 cells is consistent with all of the relevant data. We propose that a transient rise in intracellular redox potential establishes conditions permissive for differentiation to take place. Hypophosphorylated Rb will bind E2F and inhibit-activation of genes, such as c-myc, that are required for progression through the cell cycle. Reductions in Myc protein reduce E2F transcription and synthesis. After a permissive state for differentiation exists, what additional stimuli activate the differentiation response? NSC3852 inhibits histone deacetylase activity in vitro at the same concentrations used in this study, and we suggest that inhibition of histone deacetylase is a requisite step in the differentiation response to NSC2852. Furthermore, because TSA, SAHA, and SBHA also generate ROS in MCF-7 cells, ROS production coupled with histone deacetylase inhibition might be a general mechanism for inducing apoptosis and differentiation in breast cancer.

### Example 4

### In Vitro Evidence for Anti-Toxoplasma Activity of NSC3852

Based on the studies presented above, we knew that NSC3852 has tumor cell differentiation and anti-cancer properties, likely based on generation of reactive oxygen species. It was also known that NSC3852 has low histone deacetylase inhibition activity, as shown in HeLa cells. In view of its known activity as a histone deacetylase inhibitor and its activity in vivo, NSC3852 was tested for its ability to inhibit Toxoplasma activity in vitro.

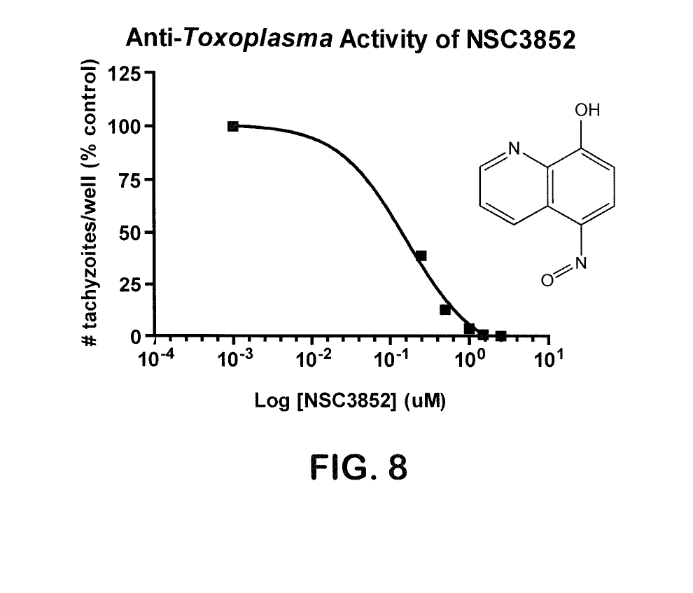

Briefly, T. gondii tachyzoites (1×10⁵) were introduced into individual wells in a 48-well culture dish containing confluent monolayers of host cells (normal human skin fibroblasts HS68). Approximately 60 hours later, the tachyzoites were harvested from the medium and counted. As can be seen from FIG. 8, the data were fit to a monophasic sigmoidal curve and the IC₅₀ was estimated from the curve using the Prism Graphpad program. A median IC₅₀ of 79 nM for NSC3852 inhibition of T. gondii tachyzoite proliferation was determined in n=5 experiments. Data points wee the average of triplicate determinations at every concentration of NSC3852 used.

Thus, the ability of yet another anti-cancer compound to treat intracellular parasites is presented here. In this experiment, a sigmoidal curve is seen for tachyzoite inhibition. This result is similar to that obtained for inhibition with scriptaid (see FIG. 2).

The evidence presented in this application enables a more precise definition of a therapeutically useful class of inhibitors of intracellular parasites, namely TSA, SAHA, and NSC3852, and by extension, other chemical structures showing structural similarity to these prototypes. It also provides a more precise definition of the amounts of compounds required to provide a treatment for intracellular parasites in vivo. While compounds of the invention may be active in many ways, we conclude that the most therapeutically useful inhibitors of intracellular parasites will target multiple essential biological processes in the parasite. One property shared by these inhibitors is the ability to decrease acetylation of a histone substrate in a nuclear extract prepared from the HeLa cell culture line derived from human carcinoma cells, generally named histone deacetylase inhibition. The second property of inhibitors that exhibit potent anti-intracellular parasite activity in vitro is the generation of reactive oxygen species (SAHA, TSA, NSC3852) thereby producing oxidative stress and DNA damage. Finally, a subset of reactive oxygen species producing histone deacetylase inhibitors (SAHA, TSA) were shown to exert in vivo activity against intracellular parasites using the murine Toxoplasmosis experimental model. Thus, although therapeutic drug development is often driven by selection of compounds that target a specific biomolecule, for example, histone deacetylase, the most active chemotherapeutic drugs frequently exhibit multiple modes of action (for example, the highly useful anti-cancer drugs 5-fluorouracil and methotrexate). A fundamental chemotherapeutic principle is that each dose of a single agent kills or growth arrests a fixed fraction of the tumor cells or infectious organisms. Therefore, values afforded by multi-targeted drugs in the treatment of intracellular parasites include: (1) a greater fraction of intracellular parasites are vulnerable to each dose of the drug treatment, (2) the emergence of drug resistance in the population of intracellular parasites will be greatly reduced, and (3) drug actions upon the intracellular parasite and upon the host cell participate in restricting parasite survival and effect pharmacologic synergism.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### REFERENCES

Bonfioli, A A and F. Orefice, Toxoplasmosis. Seminars in Opthalmology 20:129-141, 2005.
Darkin-Rattray S J, Gurnett A M, Myers R W, Dulski P M, Crumley T M, Allocco J J, Cannova C, meinke P T, Colletti S L, Bednarek M A, Singh S B, Goetz M A, Dombrowski A W, Polishook J D, and Schmatz D M, “Apicidin: a novel antiprotozoal agent that inhibits parasite histone deacetylase”, Proc. Natl. Acad. Sc (USA) 93:13143-13147, 1996.
Finnin, M. S. et al., “Structures of a histone deacetylase homologue bound to the TSA and SAHA inhibitors”, Nature 401:188-193, 1999.
Gabrielli B G, “Histone deacetylase inhibitors deliver their therapeutic benefit by targeting the cell cycle”, American Association for Cancer Research Education Book: 318-321, 1996.
Jones-Brando L, Torrey E F, and Yolken R (2003) Drugs used in the treatment of schizophrenia and bipolar disorder inhibit the replication of Toxoplasma gondii. Schizophrenia Research 62 237-244.
Kelly W K, O'Connor O A, Krug L M, Chiao J H, Heaney M, Curley T, MacGregore-Cortelli B, Tong W, Secrist J P, Schwartz L, Richardson S, Chu E, Olgac S, Marks P A, Scher H, Richon V M (2005) Phase I study of an oral histone deacetylase inhibitor, suberoylanilide hydroxamic acid, in patients with advanced cancer. J. Clin. Oncol. 23: 3923-3931.
Keen J C, Yan L, Mack K M, Pettit C, Smith D, Sharma D, and Davidson N E (2003) A novel histone deacetylase inhibitor: Scriptaid, enhances expression of functional estrogen receptor α (ER) in ER negative human breast cancer cells in combination with 5-aza 2′-deoxycytidine. Breast Cancer Res. Treat. 81:177-186.
Khan F, Tang, J, Qin C-L and Kim K (2002) Cyclin-dependent kinase TPK2 is a critical cell cycle regulator in Toxoplasma gondii. Molec. Microbiology 45:321-332.
Komatsu N, Kawamata N, Takeuchi S, Yin D, Chien W, Miller C W, and Koeffler H P (2006) SAHA, a HDAC inhibitor, has profound anti-growth activity against non-small cell lung cancer cells. Oncol. Rep. 15:187-191.
Martirosyan A., S. Leonard, X. Shi, B. Griffith, P. Gannett, and J. Strobl. 2006. Actions of a histone deacetylase inhibitor NSC3853 (5-nitroso-8-quinolinol) links reactive oxygen species to cell differentiation and apoptosis in MCF-7 human mammary tumor cells. Journal of Pharmacology and Experimental Therapeutics 317: 546-552.
Monneret, C., “Histone deacetylase inhibitors”, European Journal of Medicinal Chemistry 40:1-13, 2005.
Murray H W and Cohn Z A (1979) Macrophage oxygen-dependent anatimicrobial activity I. Susceptibility of Toxoplasma gondii to oxygen intermediates. J. Exp. Med. 150:938-949.
O'Connor O A, et al., “Clinical experience with intravenous and oral formulations of the novel histone deacetylase inhibitor suberoylanilide hydroxamic acid in patients with advanced hematologic malignancies”, J. Clin. Oncol. 24:166-173, 2006.
Radke J R, Striepen B, Guerini M N, Jerome M E, Roos D S, and White M W (2001) Defining the cell cycle for the tachyzoite stage of Toxoplasma gondii. Mol. Cell. Parasitol. 115: 165-175.
Sonnemann J, et al., “Comparative evaluation of the treatment efficacy of suberoylanilide hydroxamic acid (SAHA) and paclitaxel in ovarian cancer cell lines and primary ovarian cancer cells from patients” BMC Cancer 6:183 (Epub 2006).
Su G H, Sohn T A, Ryu B, Kern S E (2000). A novel histone deacetylase inhibitor identified by high-throughput transcriptional screening of a compound library. Cancer Res. 60: 3137-3142.

Takai N, Ueda T, Nishida M, Nasu K, and Narahar H (2006) A novel histone deacetylase inhibitor, Scriptaid, induces growth inhibition, cell cycle arrest and apoptosis in human endometrial cancer and ovarian cancer cells. Int. J. Mol. Med. 17: 323-329.
Wilson A J, Byun D-S, Popova N, Murray L B, L′Itallen K, Sowa Y, Arango D, Velcich A, Augenlicht L H, and Mariadason J M (2006) Histone deacetylase 3 (HDAC3) and other class I HDACs regulate colon cell maturation and p21 expression and are deregulated in human colon cancer. J. Biol. Chem. 281:13548-13558.
Yoshida M and Beppu T (1988) Reversible arrest of proliferation of rat 3Y1 fibroblasts in both the G1 and G2 phases by trichostatin A. Exp. Cell Res. 177:122-131.
Zhang C, Richon V, Ni X, Talpur R, and Duvic M (2005) Selective induction of apoptosis by hi stone deacetylase inhibitor SAHA in cutaneous T-cell lymphoma cells: relevance to mechanism of therapeutic action. J. Invest. Dermatol. 125:1045-1052.

1. A method of treating a subject infected or at risk of being infected with an intracellular parasite, said method comprising:
administering to the subject at least one compound capable of generating reactive oxygen species in amount sufficient to affect the growth or replication of at least one intracellular parasite within a cell of the subject.

2. The method of claim 1, wherein the compound comprises a structure that can be represented by Formula I or Formula II.

3. The method of claim 2, wherein the compound of Formula I is scriptaid.

4. The method of claim 2, wherein the compound of Formula I is suberoylanilide hydroxamic acid (SAHA).

5. The method of claim 1, which is a prophylactic method that blocks or reduces the likelihood of the subject showing clinical signs of an infection.

6. The method of claim 1, which is a therapeutic method that reduces or eliminates at least one clinical symptom of infection with an intracellular parasite.

7. The method of claim 6, wherein the method reduces the total number of intracellular parasites in the subject.

8. The method of claim 6, wherein the method eliminates the intracellular parasite from the subject.

9. The method of claim 1, wherein the subject is a human.

10. The method of claim 1, wherein the intracellular parasite is Toxoplasma gondii.

11. The method of claim 1, wherein the intracellular parasite is Cryptosporidium parvum or Cryptosporidium hominus.

12. The method of claim 1, wherein the intracellular parasite is Eimera tenella, Plasmodium falciparum, Sarcocysis neurona, Neospora hughesi, or Neospora caninum.

13. A composition for treatment of intracellular parasites, said composition comprising:
at least one compound that can cause generation of reactive oxygen species in an intracellular parasite, and
at least one other substance that is compatible with the compound and is biologically tolerable.

14. The composition of claim 13, wherein the compound comprises a structure that can be represented by Formula I or Formula II.

15. The composition of claim 14, wherein the compound of Formula I is scriptaid.

16. The composition of claim 14, wherein the compound of Formula I is suberoylanilide hydroxamic acid (SAHA).

17. The composition of claim 13, which is a pharmaceutical composition for prophylactic or therapeutic treatment of a subject infected by an intracellular parasite.

18. A method of screening for compounds that are active against one or more intracellular parasite, said method comprising:
contacting a compound known to, or suspected of being able to, produce reactive oxygen species, with an intracellular parasite, or a substance derived from an intracellular parasite, and
determining the effect of the compound on the parasite or substance derived from the parasite,
wherein an effect on the activity of the compound indicates the potential of the compound as a drug.

19. The method of claim 18, wherein the method is practice in vitro with tissue culture cells, and wherein the substance derived from an intracellular parasite is genomic DNA inside the intracellular parasite cell.

20. The method of claim 18, wherein multiple compounds are caused to contact the genomic DNA.

21. The method of claim 18, wherein the method is practiced in vivo.

22. The method of claim 18, wherein the intracellular parasite is Toxoplasma gondii, Cryptosporidium parvum, Cryptosporidium hominus, Cyclospora cayetanensis, Isospora belli, Eimera tenella, Plasmodium falciparum, Sarcocysis neurona, Neospora hughesi, or Neospora caninum.
